(19) United States
(12) Patent Application Publication
(10) Pub. No.: US 2011/0136762 A1
(43) Pub. Date: Jun. 9, 2011

(54) FUNGICIDAL PYRIDAZINES

(75) Inventor: Paula Louise Sharpe, Middletown, DE (US)

(73) Assignee: E.I. Du Pont de Nemours and Company, Wilmington, DE (US)

(21) Appl. No.: 13/056,675

(22) PCT Filed: Sep. 17, 2009
(86) PCT No.: PCT/US09/57245

§ 371 Date: Jan. 31, 2011

Related U.S. Application Data
(60) Provisional application No. 61/194,179, filed on Sep. 24, 2008.

(51) Int. Cl. A01N 43/58; C07D 237/06; C07D 403/04; A01P 3/00; C07F 7/02; A01N 55/10

(52) U.S. Cl. 514/63; 544/224; 514/247; 544/238; 514/252.03; 544/229

(57) ABSTRACT

Disclosed are compounds of Formula 1, including all geometric and stereoisomers, N-oxides, and salts thereof,

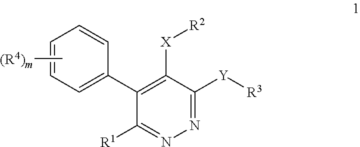

wherein
R¹, R², R³, R⁴, X, Y and m are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain pyridazines, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publication WO 2005/121104 discloses certain pyridazine derivatives of Formula i

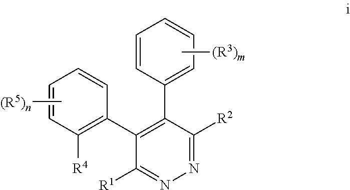

and their use as fungicides.

### SUMMARY OF THE INVENTION

This invention is directed to compounds of Formula 1 (including all geometric and stereoisomers), N-oxides, and salts thereof, agricultural compositions containing them and their use as fungicides:

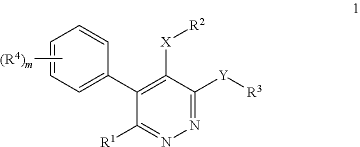

wherein
R¹ is H, halogen, cyano, hydroxy, amino, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ alkoxycarbonyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkylamino or C₂-C₄ dialkylamino;
each X and Y is independently CH₂ or a direct bond;
R² is a phenyl ring optionally substituted with up to 5 substituents independently selected from R⁵; or a 3- to 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁵ on carbon atom ring members and R^{5a} on nitrogen atom ring members;
R³ is a phenyl ring optionally substituted with up to 5 substituents independently selected from R⁶; or a 3- to 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁶ on carbon atom ring members and R^{6a} on nitrogen atom ring members;
each R⁴, R⁵ and R⁶ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₁-C₆ alkylthio, C₁-C₆ haloalkylthio, C₂-C₆ alkylcarbonylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₃-C₉ trialkylsilyl or —Z—V—W;
each Z is independently O, S(═O)ₙ, NR⁸ or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each W is independently NR^{9a}R^{9b}, OR¹⁰ or S(═O)ₙR¹⁰;
each R^{5a} and R^{6a} is independently cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₁-C₆ alkylthio, C₁-C₆ haloalkylthio, C₁-C₆ alkylsulfonyl, C₁-C₆ haloalkylsulfonyl or C₃-C₉ trialkylsilyl; or
one pair of R⁴ substituents, one pair of R⁵ or R^{5a} substituents, or one pair of R⁶ or R^{6a} substituents attached to adjacent ring atoms are each independently taken together with the atoms to which they are attached to form a 5- to 7-membered fused ring, each fused ring containing ring members selected from carbon atoms and up to 4 heteroatoms independently selected from up to 2 oxygen, up to 2 sulfur and up to 3 nitrogen atoms, and optionally substituted with up to 3 substituents independently selected from the group consisting of halogen, cyano, nitro, C₁-C₂ alkyl and C₁-C₂ alkoxy on carbon atom ring members and from the group consisting of cyano, C₁-C₂ alkyl and C₁-C₂ alkoxy on nitrogen atom ring members; or
one pair of R⁵ substituents, or one pair of R⁶ substituents attached to the same ring atom are each independently taken together with the atom to which they are attached to form a 5- to 7-membered spirocyclic ring, each spirocyclic ring containing ring members selected from carbon atoms and up to 4 heteroatoms independently selected from up to 2 oxygen, up to 2 sulfur and up to 3 nitrogen atoms, and optionally substituted with up to 3 substituents independently selected from the group consisting of halogen, cyano, nitro, C₁-C₂ alkyl and C₁-C₂ alkoxy on carbon atom ring members and from the group consisting of cyano, C₁-C₂ alkyl and C₁-C₂ alkoxy on nitrogen atom ring members;
each R⁷ is independently H or C₁-C₆ alkyl;
each R⁸ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{9a} and R^{9b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{9a} and R^{9b} attached to the same nitrogen atom are taken together with the nitrogen atom to form a 3- to 6-membered heterocyclic ring, the ring optionally substituted with up to 5 substituents independently selected from R¹¹;
each R¹⁰ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹¹ is independently halogen, C₁-C₆ alkyl, C₁-C₆ haloalkyl or C₁-C₆ alkoxy;
m is 0, 1, 2, 3, 4 or 5;
each n is independently 0, 1 or 2; and
p and q are independently 0, 1 or 2 in each instance of S(═O)ₚ(═NR⁷)_{q}, provided that the sum of p and q is 0, 1 or 2;
provided that when R² and R³ are phenyl rings, then at least one of R² and R³ is substituted with a substituent other than hydrogen.

More particularly, this invention pertains to a compound selected from Formula 1 (including all geometric and stereoisomers), an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising a mixture of a compound of Formula 1 (including all geometric and stereoisomers) or an N-oxide or a salt thereof and at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of Formula 1 (including all geometric and stereoisomers) or an N-oxide or salt thereof (e.g., as a composition described herein).

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl such as methyl, ethyl, n-propyl, i-propyl, and the different butyl, pentyl or hexyl isomers. “Alkenyl” includes straight-chain or branched alkenes, such as ethenyl, 1-propenyl, 2-propenyl, and the different butenyl, pentenyl or hexenyl isomers. “Alkenyl” also includes polyenes such as 1-propadienyl and 2,4-hexadienyl. “Alkynyl” includes straight-chain or branched alkynes such as ethynyl, 1-propynyl, 2-propynyl, and the different butynyl, pentynyl or hexynyl isomers. “Alkynyl” also includes moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃), and the different butylene, pentylene and hexylene isomers. “Alkenylene” denotes a straight-chain or branched alkenediyl containing one olefinic bond. Examples of “alkenylene” include CH═CH, CH₂CH═CH, CH═C(CH₃). “Alkynylene” denotes a straight-chain or branched alkynediyl containing one triple bond. Examples of “alkynylene” include CH₂C≡C, CCCH₂CH(CH₃), and the different butynylene, pentynylene and hexynylene isomers.

“Alkoxy” includes, for example, methoxy, ethoxy, n-propyloxy, i-propyloxy, and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃OCH₂(CH₃)CHCH₂.

“Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O), and the different butylsulfinyl, pentylsulfinyl and hexylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂, CH₃CH₂SCH₂CH₂, and other alkyl moieties bonded to sulfur, then straight-chain or branched alkyl groups; “alkylsulfinylalkyl” and “alkylsulfonylalkyl” include the corresponding sulfoxides and sulfones, respectively.

“Alkylamino” includes an NH radical substituted with straight-chain or branched alkyl. Examples of “alkylamino” include CH₃CH₂NH, CH₃CH₂CH₂NH and (CH₃)₂CHCH₂NH. Examples of “dialkylamino” include (CH₃)₂N, (CH₃CH₂CH₂)₂N and CH₃CH₂(CH₃)N.

“Cyanoalkyl” denotes an alkyl group substituted with one cyano group. Examples of “cyanoalkyl” include NCCH₂, NCCH₂CH₂ and CH₃CH(CN)CH₂. “Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, methylcyclopentyl and methylcyclohexyl. The term “cycloalkylalkyl” denotes cycloalkyl substitution on an alkyl group. Examples of “cycloalkylalkyl” include cyclopropylmethyl, cyclopentylethyl, and other cycloalkyl moieties bonded to straight-chain or branched alkyl groups. “Alkylcycloalkylalkyl” denotes alkyl substitution on a cycloalkylalkyl moiety. Examples include methylcyclohexylmethyl and ethylcyclopentylmethyl. The term “cycloalkoxy” denotes cycloalkyl linked through an oxygen atom such as cyclopentyloxy and cyclohexyloxy. “Cycloalkylcarbonyl” denotes cycloalkyl bonded to a C(═O) moiety including, for example, cyclopropylcarbonyl and cyclopentylcarbonyl. The term “cycloalkoxycarbonyl” means cycloalkoxy bonded to a C(═O) moiety, for example, cyclopropyloxycarbonyl and cyclopentyloxycarbonyl. The term “cycloalkylene” denotes a cycloalkanediyl ring. Examples of “cycloalkylene” include cyclopropylene, cyclobutylene, cyclopentylene and cyclohexylene. The term “cycloalkenylene” denotes a cycloalkenediyl ring containing one olefinic bond. Examples of “cycloalkenylene” include cylopropenediyl and cyclpentenediyl.

“Alkylcarbonyl” denotes straight-chain or branched alkyl groups bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃C(═O), CH₃CH₂OC(═O), CH₃CH₂CH₂C(═O), (CH₃)₂CHOC(═O), and the different butoxy- or pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O), and the different butylamino- or pentylaminocarbonyl isomers. Examples of “dialkylaminocarbonyl” include (CH₃)₂NC(═O), (CH₃CH₂)₂NC(═O), CH₃CH₂(CH₃)NC(═O), (CH₃)₂CHN(CH₃)C(═O) and CH₃CH₂CH₂(CH₃)NC(═O). The term “alkylcarbonyloxy” denotes straight-chain or branched alkyl bonded to a C(═O)O moiety. Examples of “alkylcarbonyloxy” include CH₃CH₂C(═O)O and (CH₃)₂CHC(═O)O.

“Alkylcarbonylthio” denotes straight-chain or branched alkylcarbonyl attached to and linked through a sulfur atom. Examples of “alkylcarbonylthio” include CH₃C(═O)S, CH₃CH₂CH₂C(═O)S and (CH₃)₂CHC(═O)S. “(Alkylthio)carbonyl” denotes a straight-chain or branched alkylthio group bonded to a C(═O) moiety. Examples of “(alkylthio)carbonyl” include CH₃SC(═O), CH₃CH₂CH₂SC(═O) and (CH₃)₂CHSC(═O). “Alkoxy(thiocarbonyl)” denotes a straight-chain or branched alkoxy group bonded to a C(═S) moiety. Examples of “alkoxy(thiocarbonyl)” include CH₃C(═S), CH₃CH₂CH₂C(═S) and (CH₃)₂CHOC(═S).

The term “halogen”, either alone or in compound words such as “haloalkyl” includes fluorine, chlorine, bromine or iodine. Further, when used in compound words such as “haloalkyl” said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “halocycloalkyl”, “haloalkoxy”, “halocycloalkoxy”, “haloalkylcarbonyl”, “haloalkylthio”, “haloalkylsulfinyl”, “haloalkylsulfonyl”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “halocycloalkyl” include 2-chlorocyclopropyl, 2-fluorocyclobutyl, 3-bromocyclopentyl and 4-chorocyclohexyl. Examples of “haloalkoxy” include CF₃O, CCl₃CH₂O, HCF₂CH₂CH₂O and CF₃CH₂O. Examples of “halocycloalkoxy” include 2-chlorocyclopentyloxy and 2-fluorocyclohexyloxy. Examples of “haloalkylcarbonyl” include CF₃C(═O), CH₃CCl₂C(═O), CCl₃CH₂CH₂C(═O) and CF₃CF₂C(═O). Examples of “haloalkylthio” include CCl₃S, CF₃S, CCl₃CH₂S and ClCH₂CH₂CH₂S. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂.

“Trialkylsilyl” includes 3 branched and/or straight-chain alkyl radicals attached to and linked through a silicon atom, such as trimethylsilyl, triethylsilyl and tert-butyldimethylsilyl.

The total number of carbon atoms in a substituent group is indicated by the “Cᵢ-Cⱼ” prefix where i and j are numbers from 1 to 9. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents, for example, (R⁴)ₘ wherein m is 1, 2, 3, 4 or 5. When a variable group is shown to be optionally attached to a position, for example, (R^{v})ᵣ in U-1 of Exhibit 1 wherein r may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 (e.g., substituents R² and R³, or a pair of R⁴ substituents taken together to form a ring system) is carbocyclic or heterocyclic. The term “ring system” denotes two or more connected rings. The term “fused” as used herein with respect to a ring system means at least two rings thereof sharing two common and adjacent atoms. The term “fused bicyclic ring system” denotes a ring system consisting of two rings sharing two common and adjacent atoms.

The term “nonaromatic” includes rings that are fully saturated as well as partially or fully unsaturated, provided that none of the rings are aromatic. The term “aromatic” indicates that each of the ring atoms of a fully unsaturated ring is essentially in the same plane and has a p-orbital perpendicular to the ring plane, and that (4n+2) π electrons, where n is a positive integer, are associated with the ring to comply with Hückel's rule.

The terms “carbocyclic ring”, “carbocycle” or “carbocyclic ring system” denote a ring or ring system wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon (e.g., N, O or S). Typically a heterocyclic ring contains no more than 2 oxygens, no more than 2 sulfurs and no more 3 nitrogens. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or aromatic heterocyclic ring. Unless otherwise indicated, heterocyclic rings and ring systems can be attached through any available carbon or nitrogen by replacement of a hydrogen on said carbon or nitrogen.

The term “ring member” refers to an atom (e.g., C, O, N or S) or other moiety (e.g., C(═O), C(═S) or S(═O)ₚ(═NR⁷)_{q}) forming the backbone of a ring or ring system.

The term “spirocyclic ring” denotes a ring connected at a single atom to another ring on Formula 1 (so the rings have a single atom in common). Illustrative of spirocyclic rings are ring systems J-1 through J-8 depicted in Exhibit 4.

As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

In the context of the present invention when an instance of R² and R³ comprises a phenyl or a 6-membered heterocyclic ring, the ortho, meta and para positions of each ring is relative to the connection of the ring to the remainder of Formula 1. Further, when an instance of R² and/or R³ comprises a phenyl or a 6-membered heterocyclic ring attached through the linker CH₂ (i.e. X and/or Y is CH₂) to the remainder of Formula 1, the ortho, meta and para positions of each ring is relative to the connection of the ring to the linker CH₂.

As noted above, each R² and R³ is, inter alia, a 3- to 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, each heterocyclic ring optionally substituted with up to 5 substituents independently selected from any substituent defined in the Summary of the Invention for R² and R³ (i.e. the R² heterocyclic ring is optionally substituted with R⁵ on carbon atom ring members and R^{5a} on nitrogen atom ring members; and the R³ heterocyclic ring is optionally substituted with R⁶ on carbon atom ring members and R^{6a} on nitrogen atom ring members). As the substituents are optional, 0 to 5 substituents may be present, limited only by the number of available points of attachment. In this definition the members of the heterocyclic ring are selected from up to 2 oxygen, up to 2 sulfur and up to 3 nitrogen atoms are optional, provided at least one ring member is not carbon (e.g., N, O or S). The definition of S(═O)ₚ(═NR⁷)_{q} allows the up to 2 sulfur ring members to be oxidized sulfur moieties (e.g., S(═O) or S(═O)₂) or unoxidized sulfur atoms (i.e. when p and q are both zero). The nitrogen atom ring members may be oxidized as N-oxides, because compounds relating to Formula 1 also include N-oxide derivatives. The up to 3 carbon atom ring members selected from C(═O) and C(═S) are in addition to the up to 4 heteroatoms selected from up to 2 oxygen, up to 2 sulfur and up to 3 nitrogen atoms.

Also, as noted above, when R² and/or R³ is a 3- to 6-membered heterocyclic ring, said ring may be saturated, partially unsaturated, or fully unsaturated. Examples of a 3- to 6-membered fully unsaturated heterocyclic ring include the rings U-2 through U-30 illustrated in Exhibit 1. In Exhibit 1 the variable R^{v} is independently selected from the group of substituents as defined in the Summary of the Invention for R² and R³ (i.e. the R² heterocyclic ring is optionally substituted with R⁵ on carbon atom ring members and R^{5a} on nitrogen atom ring members; and the R³ heterocyclic ring is optionally substituted with R⁶ on carbon atom ring members and R^{6a} on nitrogen atom ring members) and r is an integer from 0 to 5, limited by the number of available positions on each depicted ring. Note that when the attachment point between (R^{v})ᵣ and the depicted ring is illustrated as floating, (R^{v})ᵣ can be attached to any available carbon or nitrogen atom of the depicted ring. Also, when the attachment point between the depicted ring and Formula 1 is illustrated as floating, the depicted ring can be attached to the remainder of Formula 1 through any available carbon or nitrogen atom of the depicted ring by replacement of a hydrogen atom. As U-2, U-4, U-15, U-16, U-19, U-20, U-21 and U-22 have only one available position for the R^{v} substituent, for these rings r is limited to the integers 0 or 1, and r being 0 means that the ring is unsubstituted and a hydrogen is present at the position indicated by (R^{v})ᵣ.

Also noted above, each R² and R³ is independently, inter alia, a phenyl ring optionally substituted with up to 5 substituents independently selected from the group of substituents as defined in the Summary of Invention for R² and R³. An example of a phenyl ring optionally substituted with up to 5 substituents is the ring illustrated as U-1 in Exhibit 1, wherein R^{v} is independently selected from the group of substituents as defined in the Summary of the Invention for R² and R³ (i.e. the R² ring can be substituted with R⁵; and the R³ ring can be substituted with R⁶) and r is an integer from 0 to 5.

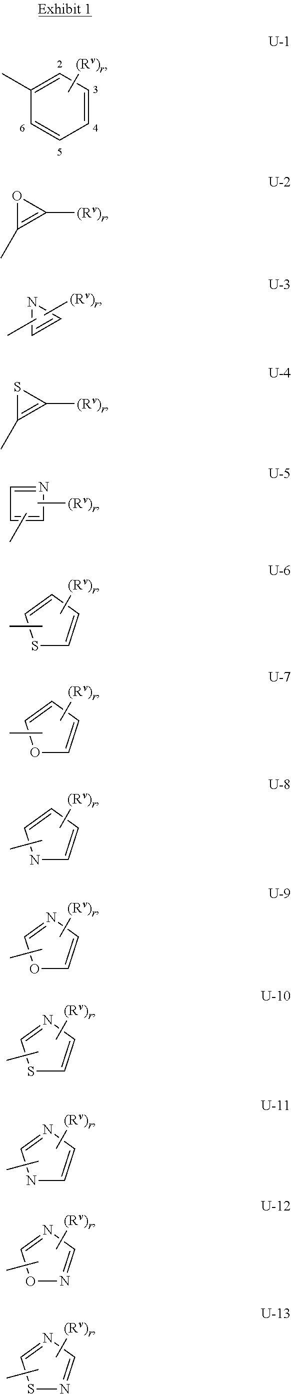

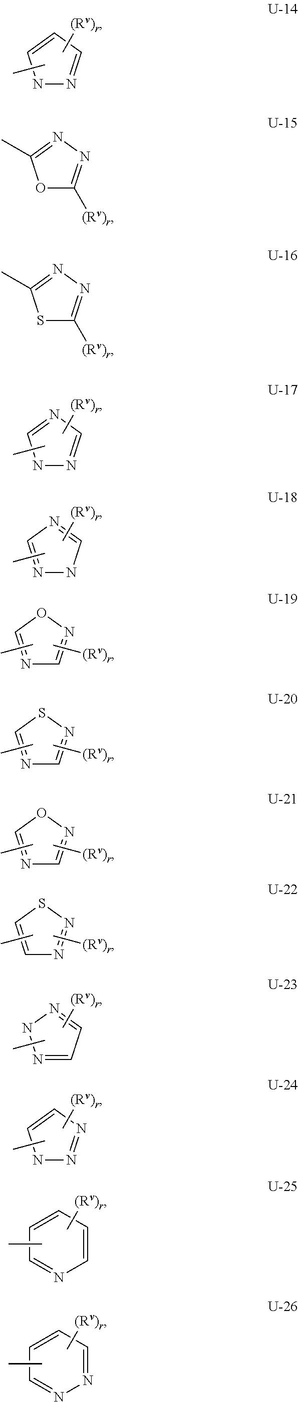

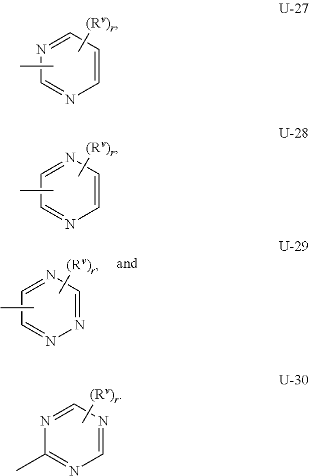

Although R^{v} groups are shown on rings U-1 through U-30, it is noted that they do not need to be present since they are optional substituents. The nitrogen atoms that require substitution to fill their valence are substituted with H or R^{v}.

Examples of a 3- to 6-membered saturated or partially unsaturated heterocyclic ring include the rings G-1 through G-44 illustrated in Exhibit 2. In Exhibit 2 the variable R^{v} is independently selected from the group of substituent as defined in the Summary of the Invention for R² and R³ (i.e. the R² heterocyclic ring is optionally substituted with R⁵ on carbon atom ring members and R^{5a} on nitrogen atom ring members; and the R³ heterocyclic ring is optionally substituted with R⁶ on carbon atom ring members and R^{6a} on nitrogen atom ring members) and r is an integer from 0 to 5, limited by the number of available positions on each depicted ring. Note that when the attachment point between (R^{v})ᵣ and the depicted ring is illustrated as floating, (R^{v})ᵣ can be attached to any available carbon or nitrogen atom of the depicted ring. Also, when the attachment point between the depicted ring and Formula 1 is illustrated as floating, the depicted ring can be attached to the remainder of Formula 1 through any available carbon or nitrogen atom of the depicted ring by replacement of a hydrogen atom.

Note that when R² and/or R³ comprises a ring selected from G-33, G-34, G-35 and G-41 through G-44, G² is O, S or N. Note that when G² is N, the nitrogen atom can complete its valence by substitution with either H or the substituents corresponding to R^{v} as defined in the Summary of Invention for R² and R³.

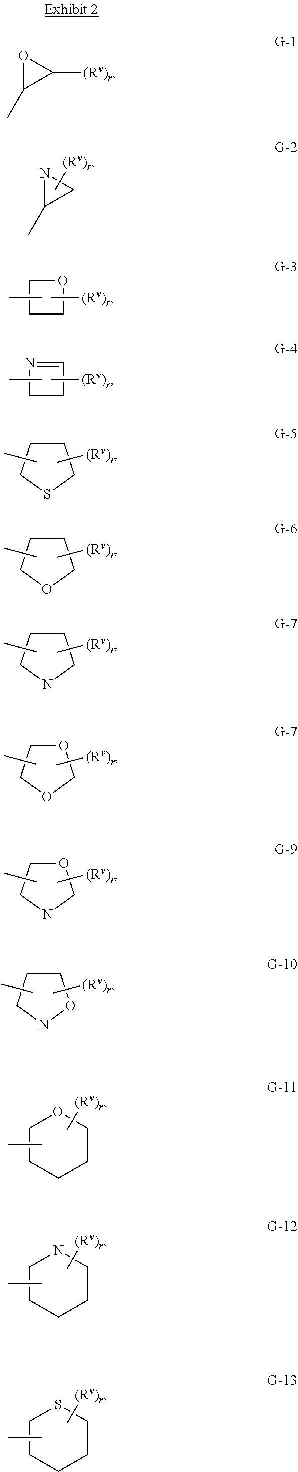

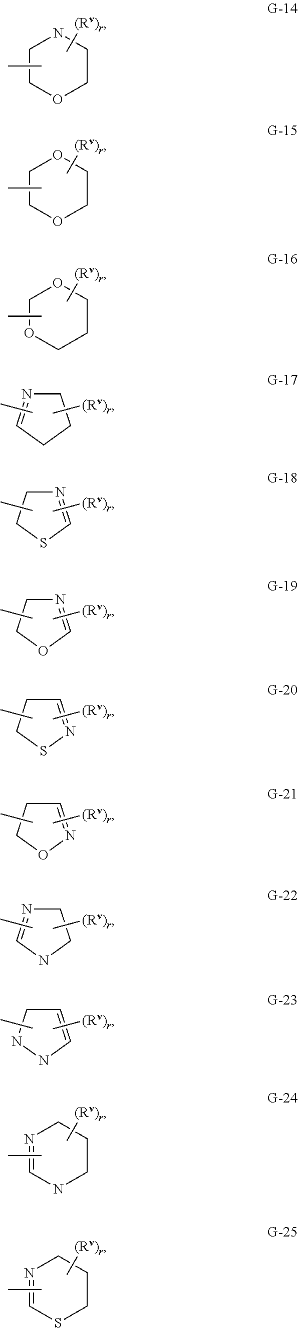

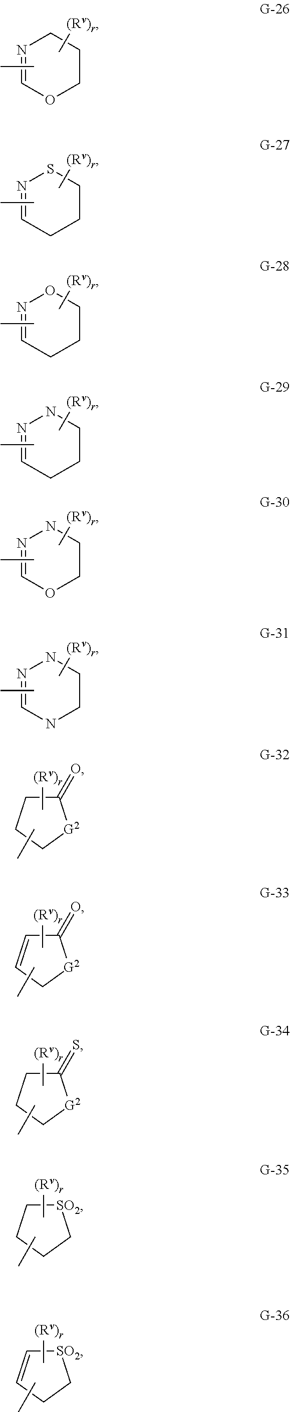

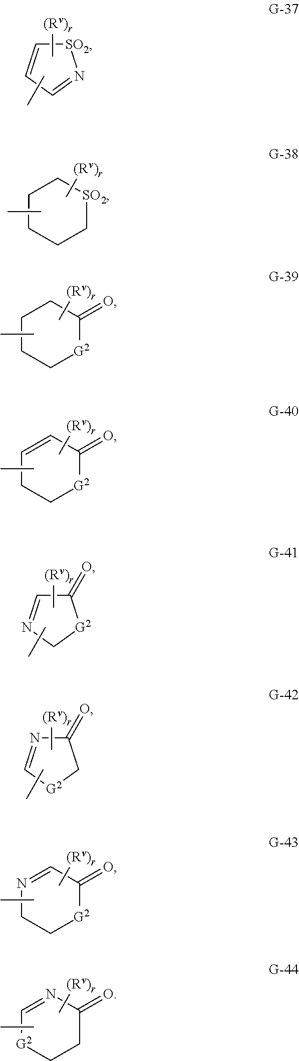

As noted in the Summary of the Invention, when a pair of R⁴ substituents are attached to adjacent ring atoms on the phenyl ring of Formula 1, or when a pair of substituents selected from R⁵ and R^{5a} substituents are attached to adjacent ring atoms on the R² ring of Formula 1, or a pair of substituents selected from R⁶ and R^{6a} substituents are attached to adjacent ring atoms on the R³ ring of Formula 1, besides the possibility of being separate substituents, they may also be connected to form a ring fused to the respective rings to which they are attached. The fused ring can be a 5- to 7-membered ring including as ring members the two atoms shared with the ring to which the substituents are attached. The other 3 to 5 ring members of the fused ring are provided by the pair of R⁴ substituents, the pair of substituents selected from R⁵ and R^{5a} substituents or the pair of substituents selected from R⁶ and R^{6a} substituents taken together. These other ring members can include up to 5 carbon atoms (as allowed by the ring size) and optionally up to 4 heteroatoms independently selected from up to 2 oxygen, up to 2 sulfur and up to 3 nitrogen atoms. The fused ring is optionally substituted with up to 3 substituents as noted in the Summary of the Invention. Exhibit 3 provides, as illustrative examples, rings formed by a pair of adjacent R⁴, R⁵, R^{5a}, R⁶ or R^{6a} substituents taken together. As these rings are fused with a ring of Formula 1, a portion of the Formula 1 ring is shown and the dashed lines represent the ring bonds of the Formula 1 ring. In certain cases, as illustrated by T-3, T-5, T-8, T-11, T-14 and T-16, the pattern of single and double bonds between ring members in the fused ring may affect the possible patterns of single and double bonds (according to valence bond theory) in the ring it is fused to in Formula 1, but each of the ring member atoms retains sp² hybridized orbitals (i.e. is able to participate in π-bonding). The rings depicted can be fused to any two adjacent atoms of a ring of Formula 1, and furthermore can be fused in either of the two possible orientations. The optional substituents (R^{v})ᵣ, are independently selected from the group consisting of halogen, cyano, nitro, C₁-C₂ alkyl and C₁-C₂ alkoxy on carbon atom ring members and from the group consisting of cyano, C₁-C₂ alkyl and C₁-C₂ alkoxy on nitrogen atom ring members. For these T-rings, r is an integer from 0 to 3, limited by the number of available positions on each depicted ring. When the attachment point between (R^{v})ᵣ and the depicted ring is illustrated as floating, R^{v} can be attached to any available carbon or nitrogen atom of the depicted ring. One skilled in the art recognizes that while r is nominally an integer from 0 to 3, some of the rings shown in Exhibit 3 have less than 3 available positions, and for those rings is limited to the number of available positions. When “r” is 0 this means the ring is unsubstituted and hydrogen atoms are present at all available positions. If r is 0 and (R^{v})ᵣ is shown attached to a particular atom, then hydrogen is attached to that atom. The nitrogen atoms that require substitution to fill their valence are substituted with H or R^{v}. Furthermore, one skilled in the art recognizes that some of the rings shown in Exhibit 3 can form tautomers, and the particular tautomer depicted is representative of all the possible tautomers.

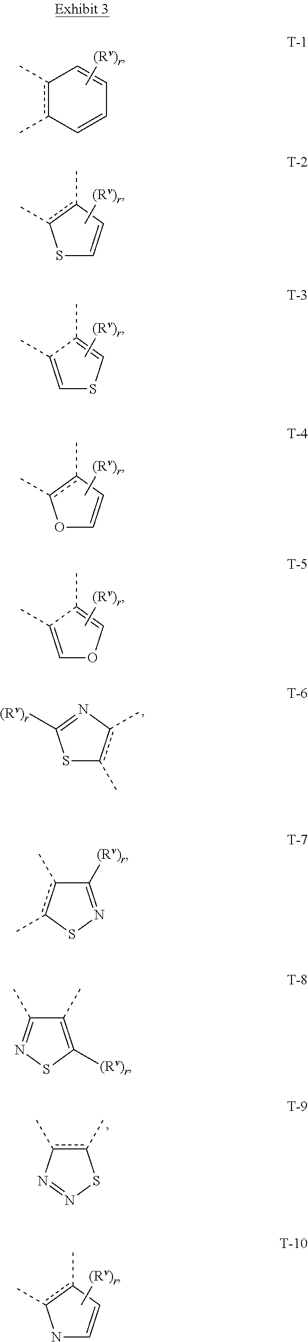

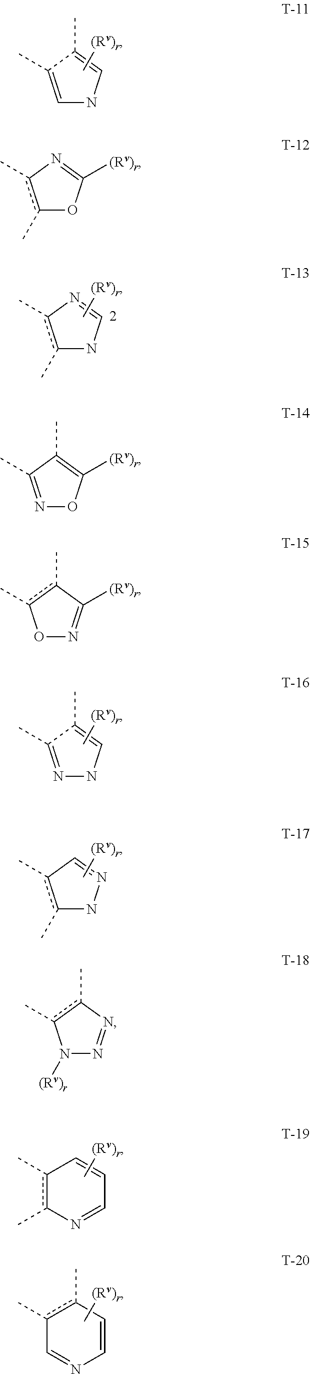

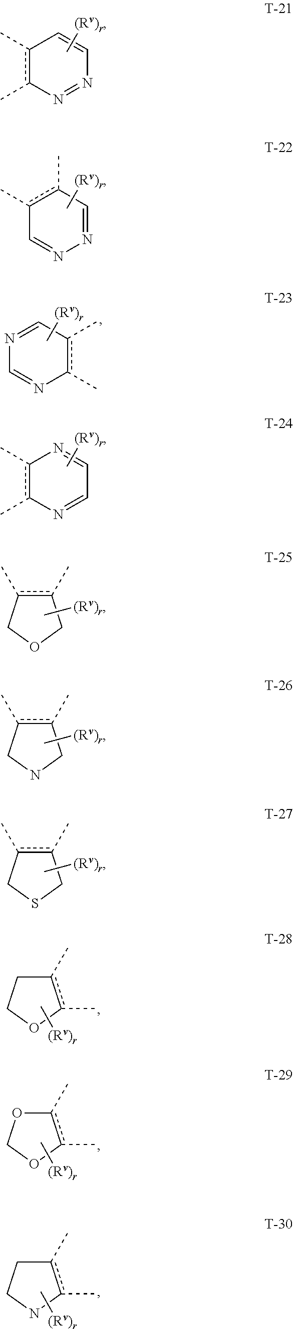

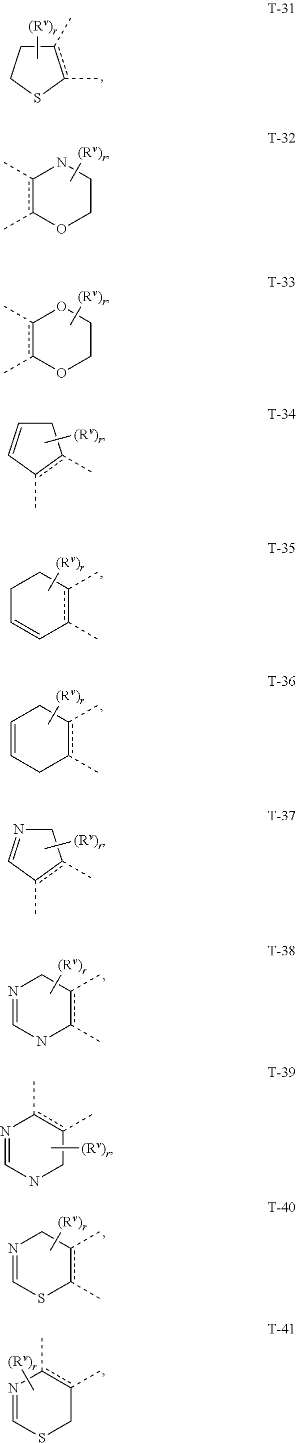

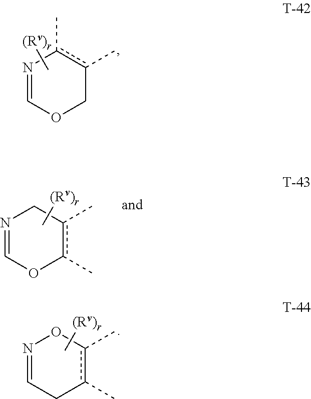

As noted in the Summary of the Invention, a pair of R⁵ or R⁶ substituents, besides the possibility being separate substituents, may also be taken together with the ring atom to which they are attached to form a 5- to 7-membered spirocyclic ring. The spirocyclic ring includes as a ring member the atom shared with the ring to which the substituents are attached. The other 4 to 6 ring members of the spirocyclic ring are provided by the pair of R⁵ substituents or the pair of R⁶ substituents taken together. Exhibit 4 provides, as illustrative examples, rings formed by a pair of R⁵ or R⁶ substituents being taken together. The dashed lines represent bonds in the ring to which the spirocyclic ring is attached. When the attachment point between (R^{v})ᵣ and the depicted ring is illustrated as floating, R^{v} can be attached to any available carbon atom of the depicted ring. The optional substituents (R^{v})ᵣ are independently selected from the group consisting of halogen, cyano, nitro, C₁-C₂ alkyl and C₁-C₂ alkoxy. When “r” is 0 this means that the ring is unsubstituted and hydrogen atoms are present at all available positions.

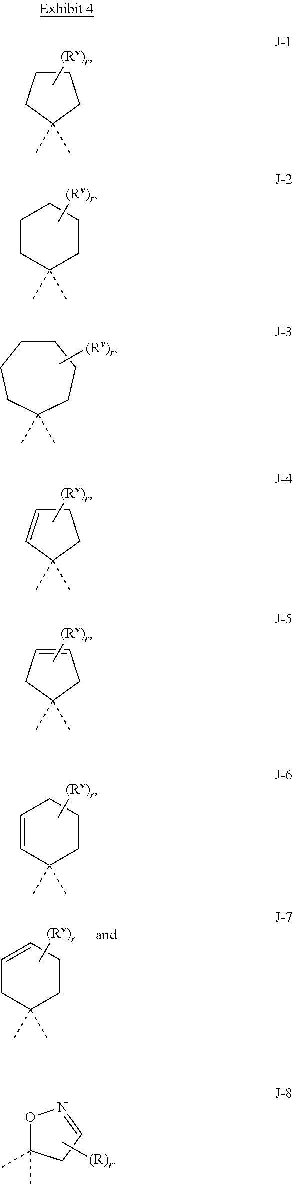

A wide variety of synthetic methods are known in the art to enable preparation of heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. Accordingly, the present invention comprises compounds selected from Formula 1, N-oxides and agriculturally suitable salts thereof.

Compounds selected from Formula 1, stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.
Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.
Embodiment 1. A compound of Formula 1 wherein R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio.
Embodiment 2. A compound of Embodiment 1 wherein R¹ is halogen, cyano, C₁-C₂ alkyl or C₁-C₂ alkoxy.
Embodiment 3. A compound of Embodiment 2 wherein R¹ is chloro, methyl or methoxy.
Embodiment 4. A compound of Embodiment 3 wherein R¹ is methyl.
Embodiment 5. A compound of Formula 1 or any one of Embodiments 1 through 4 wherein X is a direct bond.
Embodiment 6. A compound of Formula 1 or any one of Embodiments 1 through 5 wherein Y is a direct bond.
Embodiment 7. A compound of Formula 1 or any one of Embodiments 1 through 6 wherein each X and Y is a direct bond.
Embodiment 8. A compound of Formula 1 or any one of Embodiments 1 through 7 wherein R² is a phenyl ring optionally substituted with up to 5 substituents independently selected from R⁵; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁵ on carbon atom ring members and R^{5a} on nitrogen atom ring members.
Embodiment 9. A compound of Embodiment 8 wherein R² is a phenyl ring optionally substituted with up to 3 substituents independently selected from R⁵; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁵ on carbon atom ring members and R^{5a} on nitrogen atom ring members.
Embodiment 10. A compound of Embodiment 9 wherein R² is a phenyl or pyridinyl ring optionally substituted with up to 3 substituents independently selected from R⁵.
Embodiment 11. A compound of Embodiment 10 wherein R² is a pyridinyl ring attached to Formula 1 at the 3-position of the pyridinyl ring and optionally substituted with up to 3 substituents independently selected from R⁵.
Embodiment 12. A compound of Embodiment 10 wherein R² is a phenyl ring optionally substituted with up to 3 substituents independently selected from R⁵.
Embodiment 13. A compound of Embodiment 12 wherein R² is a phenyl ring optionally substituted with up to 2 substituents independently selected from R⁵.
Embodiment 14. A compound of Formula 1 or any one of Embodiments 1 through 13 wherein R³ is a phenyl ring optionally substituted with up to 5 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁶ on carbon atom ring members and R^{6a} on nitrogen atom ring members.
Embodiment 15. A compound of Embodiment 14 wherein R³ is a phenyl ring optionally substituted with up to 3 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁶ on carbon atom ring members and R^{6a} on nitrogen atom ring members.
Embodiment 16. A compound of Embodiment 15 wherein R³ is a phenyl or pyridinyl ring optionally substituted with up to 3 substituents independently selected from R⁶.
Embodiment 17. A compound of Embodiment 16 wherein R³ is a pyridinyl ring attached to Formula 1 at the 3-position of the pyridinyl ring and optionally substituted with up to 3 substituents independently selected from R⁶.
Embodiment 18. A compound of Embodiment 16 wherein R³ is a phenyl ring optionally substituted with up to 3 substituents independently selected from R⁶.
Embodiment 19. A compound of Embodiment 18 wherein R³ is a phenyl ring optionally substituted with up to 2 substituents independently selected from R⁶.
Embodiment 20. A compound of Formula 1 or any one of Embodiments 1 through 19 wherein when R² and R³ are each independently an optionally substituted phenyl or pyridinyl ring, then the R² ring is substituted with 1 to 3 substituents and the R³ ring is substituted with 0 to 2 substituents.
Embodiment 21. A compound of Embodiment 20 wherein when R² and R³ are each an optionally substituted phenyl ring, then the R² ring is substituted with 2 or 3 substituents and the R³ ring is substituted with 0 to 2 substituents.
Embodiment 22. A compound of Formula 1 or any one of Embodiments 1 through 21 wherein when R² and R³ are each an optionally substituted phenyl ring, then the R² ring is substituted with at least one substituent at a meta position and the R³ ring is substituted with at least one substituent at an ortho or para position.
Embodiment 23. A compound of Embodiment 22 wherein when R² and R³ are each an optionally substituted phenyl ring, then the R² ring is substituted with at least two substituents at the meta positions and the R³ ring is substituted with at least one substituent at an ortho or para position.
Embodiment 24. A compound of Formula 1 or any one of Embodiments 1 through 23 wherein each R⁴, R⁵ and R⁶ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio, C₁-C₆ haloalkylthio or —Z—V—W.
Embodiment 25. A compound of Embodiment 24 wherein each R⁴, R⁵ and R⁶ is independently halogen, C₁-C₆ alkyl, C₂-C₆ alkenyl, C₁-C₆ haloalkyl, C₁-C₆ alkoxy or —Z—V—W.
Embodiment 26. A compound of Embodiment 25 wherein each R⁴, R⁵ and R⁶ is independently halogen, C₁-C₃ alkyl, C₂-C₃ alkenyl, C₁-C₃ haloalkyl or C₁-C₃ alkoxy.
Embodiment 27. A compound of Embodiment 26 wherein each R⁴, R⁵ and R⁶ is independently halogen, C₁-C₃ alkyl or C₁-C₃ alkoxy.
Embodiment 28. A compound of Embodiment 27 wherein each R⁴, R⁵ and R⁶ is independently halogen, methyl or methoxy.
Embodiment 29. A compound of Embodiment 28 wherein each R⁴, R⁵ and R⁶ is independently Cl, F, methyl or methoxy.
Embodiment 30. A compound of Embodiment 29 wherein each R⁴ and R⁵ is independently Cl, F or methoxy.
Embodiment 31. A compound of Embodiment 30 wherein each R⁵ is methoxy.
Embodiment 32. A compound of Formula 1 or any one of Embodiments 1 through 25 wherein each Z is independently O or NR⁸.
Embodiment 33. A compound of Embodiment 32 wherein each Z is independently O or NH.
Embodiment 34. A compound of Embodiment 33 wherein each Z is O.
Embodiment 35. A compound of Formula 1 or any one of Embodiments 1 through 25 or Embodiments 32 through 34 wherein each V is C₂-C₄ alkylene.
Embodiment 36. A compound of Formula 1 or any one of Embodiments 1 through 25 or Embodiments 32 through 35 wherein each W is independently NR^{9a}R^{9b} or OR¹⁰.
Embodiment 37. A compound of Formula 1 or any one of Embodiments 1 through 25 or Embodiments 32 through 36 wherein each R^{9a} and R^{9b} is independently H, C₁-C₆ alkyl or C₁-C₆ haloalkyl.
Embodiment 38. A compound of Embodiment 37 wherein each R^{9a} and R^{9b} is independently H, C₁-C₂ alkyl or C₁-C₂ haloalkyl.
Embodiment 39. A compound of Embodiment 38 wherein each R^{9a} and R^{9b} is independently H or methyl.
Embodiment 40. A compound of Formula 1 or any one of Embodiments 1 through 25 or Embodiments 34 through 39 wherein each R¹⁰ is independently H, C₁-C₆ alkyl or C₁-C₆ haloalkyl.
Embodiment 41. A compound of Embodiment 40 wherein each R¹⁰ is methyl.
Embodiment 42. A compound of Formula 1 or any one of Embodiments 1 through 41 wherein m is 0, 1, 2 or 3.
Embodiment 43. A compound of Formula 1 or any one of Embodiments 1 through 42 wherein m is 3.
Embodiment 44. A compound of Formula 1 or any one of Embodiments 1 through 43 wherein m is 3 and the R⁴ substituents are at the para and ortho positions.
Embodiment 45. A compound of Formula 1 or any one of Embodiments 1 through 44 wherein each R^{5a} and R^{6a} is independently C₁-C₃ alkyl or C₁-C₃ haloalkyl.
Embodiment 46. A compound of Embodiment 45 wherein each R^{5a} and R^{6a} is methyl.

Embodiments of this invention, including Embodiments 1-46 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-46 above as well as any other embodiments described herein, and any combination thereof, pertain to the compositions and methods of the present invention.

Combinations of Embodiments 1-46 are illustrated by:
Embodiment A1. A compound of Formula 1 wherein
   R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio;
   R² is a phenyl ring optionally substituted with up to 3 substituents independently selected from R⁵; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁵ on carbon atom ring members and R^{5a} on nitrogen atom ring members;
   R³ is a phenyl ring optionally substituted with up to 3 substituents independently selected from R⁶; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁶ on carbon atom ring members and R^{6a} on nitrogen atom ring members;
   each R⁴, R⁵ and R⁶ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio, C₁-C₆ haloalkylthio or —Z—V—W;
   each R^{5a} and R^{6a} is independently C₁-C₃ alkyl or C₁-C₃ haloalkyl; and
   m is 0, 1, 2 or 3.
Embodiment A2. A compound of Embodiment A1 wherein
   R¹ is halogen, cyano, C₁-C₂ alkyl or C₁-C₂ alkoxy;
   R² is a phenyl or pyridinyl ring optionally substituted with up to 3 substituents independently selected from R⁵;
   R³ is a phenyl or pyridinyl ring optionally substituted with up to 3 substituents independently selected from R⁶;
   each R⁴, R⁵ and R⁶ is independently halogen, C₁-C₆ alkyl, C₂-C₆ alkenyl, C₁-C₆ haloalkyl, C₁-C₆ alkoxy or —Z—V—W;
   each X and Y is a direct bond;
   each Z is independently O or NH;
   each V is C₂-C₄ alkylene;
   each W is independently NR^{9a}R^{9b} or OR¹⁰;
   each R^{9a} and R^{9b} is independently H, C₁-C₂ alkyl or C₁-C₂ haloalkyl; and
   each R¹⁰ is methyl.
Embodiment A3. A compound of Embodiment A2 wherein
   R¹ is chloro, methyl or methoxy;
   R² is a phenyl ring optionally substituted with up to 3 substituents independently selected from R⁵;
   R³ is a phenyl ring optionally substituted with up to 3 substituents independently selected from R⁶;
   each R⁴, R⁵ and R⁶ is independently halogen, C₁-C₃ alkyl, C₂-C₃ alkenyl, C₁-C₃ haloalkyl or C₁-C₃ alkoxy; and
   the R² ring is substituted with at least one substituent at a meta position and the R³ ring is substituted with at least one substituent at an ortho or para position.
Embodiment A4. A compound of Embodiment A3 wherein each R⁴, R⁵ and R⁶ is independently halogen, C₁-C₃ alkyl or C₁-C₃ alkoxy.
Embodiment A5. A compound of Embodiment A4 wherein
   each R⁴ and R⁵ is independently Cl, F or methoxy; and
   the R² ring is substituted with at least two substituents at the meta positions and the R³ ring is substituted with at least one substituent at an ortho or para position.

Specific embodiments include compounds of Formula 1 selected from the group consisting of:
4-(3,5-dimethoxyphenyl)-3-(2-fluorophenyl)-6-methyl-5-(2,4,6-trifluorophenyl)pyridazine;
4-(2,6-difluoro-4-methoxyphenyl)-5-(3,5-dimethoxyphenyl)-6-(2-fluorophenyl)-3-methylpyridazine;
4-(2-chloro-3,5-dimethoxyphenyl)-3-(2-fluorophenyl)-6-methyl-5-(2,4,6-trifluorophenyl)pyridazine;
4-(3,5-dimethoxyphenyl)-3-(2-fluorophenyl)-6-methoxy-5-(2,4,6-trifluorophenyl)pyridazine;
4-(3,5-dimethoxyphenyl)-3-(2-fluorophenyl)-5-(4-fluorophenyl)-6-methylpyridazine;
4-(3,5-dimethoxyphenyl)-5-(4-methoxyphenyl)-6-methyl-3-phenylpyridazine;
3-(2,4-difluorophenyl)-4-(3,5-dimethoxyphenyl)-5-(4-methoxyphenyl)-6-methylpyridazine;
4-(2,4-difluorophenyl)-5-(3,5-dimethoxyphenyl)-6-(2-fluorophenyl)-3-methylpyridazine;
3-chloro-4-(2,4-difluorophenyl)-5-(3,5-dimethoxyphenyl)-6-(2-fluorophenyl)pyridazine;
3-(2-fluorophenyl)-4-(3-fluorophenyl)-6-methyl-5-(2,4,6-trifluorophenyl)pyridazine;
3-[4-[5-(3,5-dimethoxyphenyl)-6-(2-fluorophenyl)-3-methyl-4-pyridazinyl]-3,5-difluorophenoxy]-N-methyl-1-propanamine;
4-(3,5-dimethoxyphenyl)-6-methyl-3-phenyl-5-(2,4,6-trifluorophenyl)pyridazine;
4-(2-chloro-3,5-dimethoxyphenyl)-6-methyl-3-phenyl-5-(2,4,6-trifluorophenyl)pyridazine;
5-(2,6-difluoro-4-methoxyphenyl)-4-(3,5-dimethoxyphenyl)-6-methyl-3-phenylpyridazine; and
4-(3,5-dimethoxyphenyl)-3-(2-fluorophenyl)-5-(4-methoxyphenyl)-6-methylpyridazine.

Embodiments of the present invention also include:
Embodiment B1. A compound of Formula 1 wherein R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio.
Embodiment B2. A compound of Embodiment B1 wherein R¹ is halogen, cyano or C₁-C₂ alkyl.
Embodiment B3. A compound of Embodiment B2 wherein R¹ is chloro or methyl.
Embodiment B4. A compound of Formula 1 or any one of Embodiments B1 through B3 wherein X is direct bond.
Embodiment B5. A compound of Formula 1 or any one of Embodiments B1 through B4 wherein Y is direct bond.
Embodiment B6. A compound of Formula 1 or any one of Embodiments B1 through B5 wherein R² is a phenyl ring optionally substituted with up to 5 substituents independently selected from R⁵; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents selected from R⁵ on carbon atom ring members and R^{5a} on nitrogen atom ring members.
Embodiment B7. A compound of Embodiment B6 wherein R² is a phenyl ring optionally substituted with up to 3 substituents independently selected from R⁵; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents selected from R⁵ on carbon atom ring members and R^{5a} on nitrogen atom ring members.
Embodiment B8. A compound of Embodiment B7 wherein R² is a phenyl ring optionally substituted with up to 3 substituents independently selected from R⁵.
Embodiment B9. A compound of Embodiment B8 wherein the R⁵ substituents are at the 2-, 3- and/or 5-positions.
Embodiment B10. A compound of Embodiment B9 wherein R² is a phenyl ring substituted with 3 substituents independently selected from R⁵.
Embodiment B11. A compound of Embodiment B8 or B9 wherein R² is a phenyl ring optionally substituted with up to 2 substituents independently selected from R⁵.
Embodiment B12. A compound of Embodiment B11 wherein the R⁵ substituents are at the 3- and/or 5-positions or at the 2- and/or 5-positions.
Embodiment B13. A compound of Embodiment B12 wherein the R⁵ substituents are at the 3- and/or 5-positions.
Embodiment B14. A compound of Embodiment B12 wherein the R⁵ substituents are at the 2- and/or 5-positions.
Embodiment B15. A compound of any one of Embodiments B11 through B14 wherein R² is a phenyl ring substituted with 2 substituents independently selected from R⁵.
Embodiment B16. A compound of Formula 1 or any one of Embodiments B1 through B15 wherein R³ is a phenyl ring optionally substituted with up to 5 substituents independently selected from R⁶; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁶ on carbon atom ring members and R^{6a} on nitrogen atom ring members.
Embodiment B17. A compound of Embodiment B16 wherein R³ is a phenyl ring optionally substituted with up to 3 substituents independently selected from R⁶; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁶ on carbon atom ring members and R^{6a} on nitrogen atom ring members.
Embodiment B18. A compound of Embodiment B17 wherein R³ is a phenyl ring optionally substituted with up to 3 substituents independently selected from R⁶.
Embodiment B19. A compound of Formula 1 or any one of Embodiments B1 through B18 wherein each R⁴, R⁵ and R⁶ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.
Embodiment B20. A compound of Embodiment B19 wherein each R⁴, R⁵ and R⁶ is independently halogen, C₁-C₆ alkyl, C₂-C₆ alkenyl, C₁-C₆ haloalkyl or C₁-C₆ alkoxy.
Embodiment B21. A compound of Embodiment B20 wherein each R⁴, R⁵ and R⁶ is independently halogen, C₁-C₆ alkyl or C₁-C₆ alkoxy.
Embodiment B22. A compound of Embodiment B21 wherein each R⁴, R⁵ and R⁶ is independently halogen, methyl or methoxy.
Embodiment B23. A compound of Embodiment B22 wherein each R⁴ is independently halogen.
Embodiment B24. A compound of Formula 1 or any one of Embodiments B1 through B23 wherein m is 3 and the R⁴ substituents are at the 2-, 4- and 6-positions.
Embodiment B25. A compound of Formula 1 or any one of Embodiments B1 through B24 wherein each R⁵ is independently halogen or methoxy.
Embodiment B26. A compound of Embodiment B25 wherein each R⁵ is methoxy.
Embodiment B27. A compound of Formula 1 or any one of Embodiments B1 through B26 wherein each R^{5a} and R^{6a} is independently C₁-C₃ alkyl or C₁-C₃ haloalkyl.
Embodiment B28. A compound of Embodiment B27 wherein each R^{5a} and R^{6a} is independently C₁-C₃ alkyl.
Embodiment B29. A compound of Embodiment B28 wherein each R^{5a} and R^{6a} is methyl.
Embodiment B30. A compound of Formula 1 or any one of Embodiments B1 through B11 and B14 through B18 wherein when one pair of R⁴ substituents, one pair of R⁵ or R^{5a} substituents, or one pair of R⁶ or R^{6a} substituents attached to adjacent ring atoms are taken together with the atoms to which they are attached to form an optionally substituted fused ring, said fused ring is 5- or 6-membered, contains ring members selected from carbon atoms and is optionally substituted with up to 3 substituents independently selected from the group consisting of C₁-C₂ alkyl and halogen on carbon ring members and from the group consisting of C₁-C₂ alkyl on nitrogen ring members.
Embodiment B31. A compound of Formula 1 or any one of Embodiments B1 through B30 wherein R⁷ is H or methyl.
Embodiment B32. A compound of Formula 1 or any one of Embodiments B1 through B30 wherein q is 0 in each instance of S(═O)ₚ(═NR⁷)_{q}.
Embodiment B33. A compound of Formula 1 or any one of Embodiments B1 through B32 wherein p is 0 in each instance of S(═O)ₚ(═NR⁷)_{q}.
Embodiment B34. A compound of Formula 1 or any one of Embodiments B1 through B30 wherein the sum of q and p is 0 in each instance of S(═O)ₚ(═NR⁷)_{q}.

Combinations of Embodiments B1-B34 are illustrated by:
Embodiment C1. A compound of Formula 1, or an N-oxide or salt thereof, wherein
   R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio;
   R² is a phenyl ring optionally substituted with up to 5 substituents independently selected from R⁵; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents selected from R⁵ on carbon atom ring members and R^{5a} on nitrogen atom ring members;
   R³ is a phenyl ring optionally substituted with up to 5 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents selected from R⁶ on carbon atom ring members and R^{6a} on nitrogen atom ring members;
   each R⁴, R⁵ and R⁶ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio;
   each R^{5a} and R^{6a} is independently C₁-C₃ alkyl or C₁-C₃ haloalkyl; and
   q is 0 in each instance of S(═O)ₚ(═NR⁷)_{q}.
Embodiment C2. A compound of Embodiment C1 wherein
   R² is a phenyl ring optionally substituted with up to 3 substituents independently selected from R⁵; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents selected from R⁵ on carbon atom ring members and R^{5a} on nitrogen atom ring members;
   R³ a phenyl ring optionally substituted with up to 3 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents selected from R⁶ on carbon atom ring members and R^{6a} on nitrogen atom ring members;
   each R^{5a} and R^{6a} is independently C₁-C₃ alkyl; and
   p is 0 in each instance of S(═O)ₚ(═NR⁷)_{q}.
Embodiment C3. A compound of Embodiment C2 wherein
   X is a direct bond;
   Y is a direct bond;
   R² is a phenyl ring optionally substituted with up to 3 substituents independently selected from R⁵;
   R³ is a phenyl ring optionally substituted with up to 3 substituents independently selected from R⁶; and
   each R⁴, R⁵ and R⁶ is independently halogen, C₁-C₆ alkyl, C₂-C₆ alkenyl, C₁-C₆ haloalkyl or C₁-C₆ alkoxy.
Embodiment C4. A compound of Embodiment C3 wherein each R⁴, R⁵ and R⁶ is independently halogen, C₁-C₆ alkyl or C₁-C₆ alkoxy.
Embodiment C5. A compound of Embodiment C4 wherein
   each R⁴ is independently halogen; and
   each R⁵ is independently halogen or methoxy.
Embodiment C6. A compound of Embodiment C5 wherein
   R² is a phenyl ring substituted with 3 substituents independently selected from R⁵, and the R⁵ substituents are at the 2-, 3- and 5-positions.
Embodiment C7. A compound of Embodiment C6 wherein
   R² is a phenyl ring substituted with 2 substituents independently selected from R⁵; and the R⁵ substituents are at the 3- and 5-positions or at the 2- and 5-positions.

Of note are compounds of Formula 1 including all stereoisomers, N-oxides, and salts thereof (including but not limited to Embodiments 1-46, A1-A5, B1-B34 and C1-C7 above) wherein each R⁴, R⁵ and R⁶ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ cyanoalkyl, C₁-C₆ hydroxyalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₁-C₆ alkylthio, C₁-C₆ haloalkylthio, C₂-C₆ alkylcarbonylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino or C₃-C₉ trialkylsilyl.

This invention provides a fungicidal composition comprising a compound of Formula 1 (including all geometric and stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as an embodiment of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-9 can be used to prepare the compounds of Formula 1. The definitions of R¹, R², R³, R⁴, X, Y and m in the compounds of Formulae 1-18 below are as defined above in the Summary of the Invention unless otherwise noted. Compounds of Formulae Ia and Ib are various subsets or analogs of the compounds of Formula 1, and all substituents for Formulae Ia and Ib are as defined above for Formula 1. Formulae 5a and 5b are subsets of Formula 5.

As shown in Scheme 1, compounds of Formula 1 can be synthesized from compounds of Formula 2 wherein Lg is a leaving group such as halogen (e.g., Cl, Br, I), sulfonate (e.g., OS(O)₂CH₃, OS(O)₂CF₃, OS(O)₂Ph-p-CH₃), or the like, using various coupling reagents in conjunction with a transition metal catalyst. In particular, compounds of Formula 2 can be contacted with compounds of Formula 3 in the presence of a palladium, copper, nickel or iron catalyst to produce compounds of Formula 1 wherein Y is CH₂ or a direct bond and R³ is an optionally substituted phenyl or heterocyclic ring bonded through carbon. In this method compounds of Formula 3 are organoboronic acids (e.g., M¹ is B(OH)₂), organoboronic esters (e.g., M¹ is B(—OC(CH₃)₂C(CH₃)₂O—)), organotrifluoroborates (e.g., M¹ is BF₃K), organotin reagents (e.g., M¹ is Sn(n-Bu)₃, Sn(Me)₃), Grignard reagents (e.g., M¹ is MgX¹) or organozinc reagents (e.g., M¹ is ZnX¹) wherein X¹ is Br or Cl. Suitable transition metal catalysts include, but are not limited to: palladium(II) acetate, palladium(II) chloride, tetrakis(triphenylphosphine)palladium(0), bis(triphenylphosphine)palladium(II) dichloride, dichloro[1,1′-bis(diphenylphosphino)ferrocene]palladium(II), bis(triphenyl-phosphine)dichloronickel(II) and copper(I) salts (e.g., copper(I) iodide, copper(I) bromide, copper(I) chloride, copper(I) cyanide or copper(I) triflate). Optimal conditions for each reaction will depend upon the catalyst used and the counterion attached to the compound of Formula 3 (i.e. M¹), as is understood by one skilled in the art. In some cases the addition of a ligand such as a substituted phosphine or a substituted bisphosphinoalkane promotes reactivity. Also, the presence of a base (such as an alkali carbonate, tertiary amine or alkali fluoride) is typically necessary for reactions involving compounds of Formula 3 where M¹ is a boronic acid or organotrifluoroborate. For reviews of this type of reaction see: E. Negishi, Handbook of Organopalladium Chemistry for Organic Synthesis, John Wiley and Sons, Inc., New York, 2002; N. Miyaura, Cross-Coupling Reactions: A Practical Guide, Springer, New York, 2002; H. C. Brown et al., Organic Synthesis via Boranes, Vol. 3, Aldrich Chemical Co., Milwaukee, Wis., 2002; Suzuki et al., Chemical Review 1995, 95, 2457-2483 and Molander et al., Accounts of Chemical Research 2007, 40, 275-286.

Compounds of Formula 1 wherein Y is a direct bond and R³ is a N-linked heterocyclic ring can be prepared via a cross-coupling reaction of compounds of Formula 2 and compounds of Formula 4. Typical reaction conditions involve the presence of a base (e.g., NaOt-Bu, K₂CO₃, K₃PO₄ or Cs₂CO₃), a palladium, nickel or copper catalyst (e.g., tris(dibenzylideneacetone)dipalladium, palladium(II) acetate, bis(1,5-cyclooctadiene)nickel or copper(I) iodide) and optionally a ligand (e.g., 1,1′-bis(diphenylphosphino)ferrocene, 1,3-bis(diphenylphosphino)propane, 2,2′-bis(diphenylphosphino)-1,1′-binaphthalene, 1,1′-bi-naphthalene-2,2′-diol or 1,1,1-tris(hydroxymethyl)ethane) in a solvent such as methanol, acetonitrile or N,N-dimethylformamide at a temperature ranging from about room temperature to the reflux temperature of the solvent. For relevant literature references, see: for example, Chen et al., Organic Letters 2006, 8, 5609-5612; Hartwig, Angew. Chem. Int. Ed. 1998, 37(15), 2046-2067 and Buchwald et al., Accounts of Chemical Research 1998, 31(12), 805-818.

One skilled in the art will appreciate that the leaving group Lg attached to compounds of Formula 2 should be selected in view of the relative reactivity of other functional groups present on Formula 2 (i.e. R¹, XR² and R⁴), so that the group Lg is displaced and not the functional groups to give the final compounds of Formula 1. The method of Scheme 1 is illustrated by Example 1, Step E and Example 2, Step E.

Compounds of Formulae 3 and 4 are commercially available and can be prepared by a wide variety of general methods known in the art.

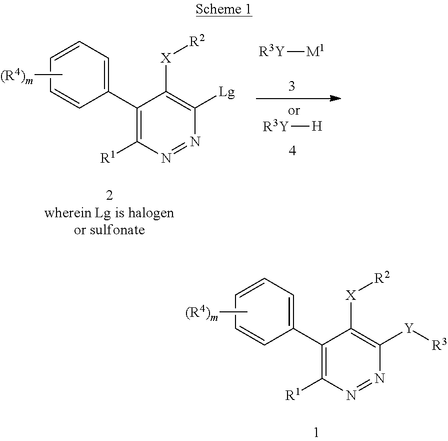

Alternatively, compounds of Formula 1 wherein R¹ is halogen, haloalkyl, or the like can be prepared by the two-step synthesis outlined in Scheme 2. In the first step, compounds of Formula Ia (Formula 1 wherein R¹ is H, alkyl, or the like prepared by the method of Scheme 1) are converted to their N-oxides of Formula Ib by treatment with an oxidizing reagent such as m-chloroperbenzoic acid (MCPBA) in an appropriate solvent such as chloroform or dichloromethane at a temperature ranging from about 0° C. to room temperature (e.g., 20° C.). Example 3 illustrates the oxidation method of Scheme 2.

Subsequent treatment of a compound of Formula Ib with a halogenating reagent results in substitution of hydrogen accompanied by loss of the oxide group to provide Formula 1 wherein R¹ is halogen, haloalkyl, or the like. Suitable halogenating reagents include phosphorus oxyhalides, phosphorus trihalides, phosphorus pentahalides, thionyl chloride, oxalyl chloride, phenylphosphonic dichloride and phosgene. Phosphorus oxyhalides are particularly useful. Suitable solvents for this reaction include, for example, dichloromethane, chloroform, chlorobutane, benzene, xylenes, chlorobenzene, tetrahydrofuran, p-dioxane, acetonitrile, and the like. In many cases the reaction can be carried out without solvent other than the compound of Formula Ib and the halogenating reagent. Example 5 illustrates the synthesis of a compound of Formula 1 wherein R¹ is chloromethyl from the corresponding compound of Formula 1 wherein R¹ is methyl.

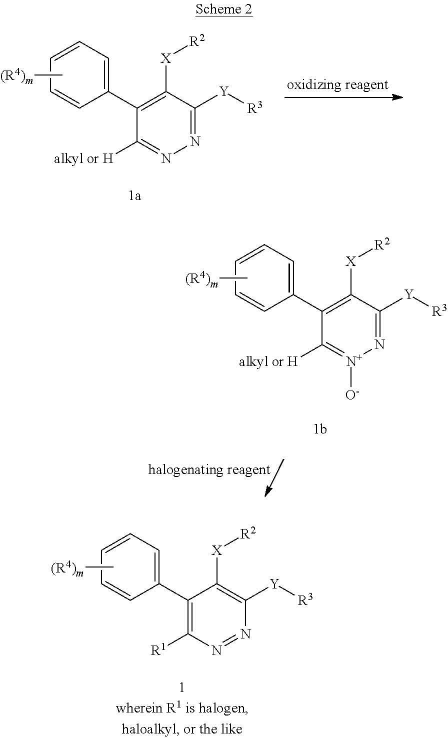

Compounds of Formula 1 wherein R¹ is halogen can be subjected to various nucleophilic and metallation reactions (using methods analogous to those already described for Scheme 1) to add substituents or modify existing substituents, and thus provide other functionalized compounds of Formula 1. For example, compounds of Formula 1 wherein R¹ is alkyl, alkenyl, alkynyl, or the like can be synthesized from corresponding compounds of Formula 1 wherein R¹ is halogen (e.g., Cl, Br or I), using various boronic acids in conjunction with a palladium catalyst. Example 7 illustrates the synthesis of a compound of Formula 1 wherein R¹ is methyl from the corresponding compound of Formula 1 wherein R¹ is chloro.

As shown in Scheme 3, compounds of Formula 2 wherein Lg is halogen (e.g., Br, Cl or I) can be prepared from corresponding pyridazinones of Formula 5 by treatment with a halogenating reagent. Suitable halogenating reagents include phosphorus oxyhalides, phosphorus trihalides, phosphorus pentahalides, thionyl chloride, oxalyl chloride, phenylphosphonic dichloride and phosgene. Phosphorus oxyhalides are particularly useful. Suitable solvents for this reaction include, for example, dichloromethane, chloroform, chlorobutane, benzene, xylenes, chlorobenzene, tetrahydrofuran, p-dioxane, acetonitrile, and the like. In many cases the reaction can be carried out without solvent other than the compound of Formula 5 and the halogenating reagent. Optionally, an organic base such as triethylamine, pyridine, N,N-dimethylaniline, and the like can be added. Addition of a catalyst such as N,N-dimethylformamide is also an option. Typical reaction temperatures range from about room temperature (e.g., 20° C.) to 200° C. For representative procedures see Czarnocki et al., Synthesis 2006, 17, 2855-2864; Brana et al., Journal of Medicinal Chemistry 2005, 48, 6843-6854; Liu et al., Journal of Medicinal Chemistry 2007, 50, 3086-3100 and Chan et al., Journal of Medicinal Chemistry 2005, 48, 4420-4431. The method of Scheme 2 is illustrated in Example 1, Step D and Example 2, Step D

Compounds of Formula 2 wherein Lg is a sulfonate (e.g., OS(O)₂CH₃, OS(O)₂CF₃, OS(O)₂Ph-p-CH₃) can also be prepared from pyridones of Formula 5 by treatment with a sulfonating reagent such as methanesulfonyl chloride, p-toluenesulfonyl chloride, trifluoromethanesulfonic anhydride or N-phenyltrifluoromethanesulfonimide. The reaction is typically run in the presence of a solvent and a base. Suitable solvents include dichloromethane, tetrahydrofuran, acetonitrile, and the like. Suitable bases include tertiary amines (e.g., triethylamine, N,N-diisopropylethylamine) and potassium carbonate. The reaction is typically conducted at a temperature between about −50° C. and the boiling point of the solvent. For references describing this general method see, for example, Markus et al., Heterocycles 1996, 43(7), 1459-1464 and Takenari et al., Chemical & Pharmaceutical Bulletin 1966, 14(10), 1074-1081.

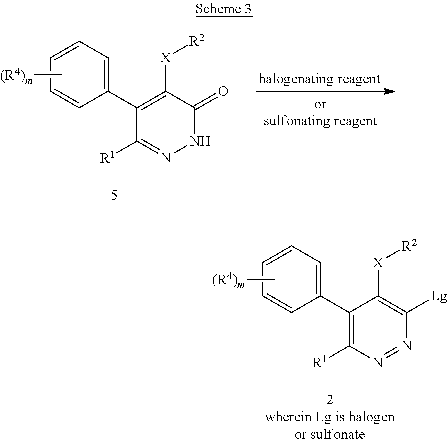

Compounds of Formula 5 can be synthesized by condensation of furanones of Formula 6 with hydrazine hydrate. The reaction is typically run in a lower alkanol solvent, such as methanol, ethanol or n-butanol at a temperature ranging from about room temperature to the reflux temperature of the solvent. For conditions and variations of this reaction see the following references: PCT Patent Application Publications WO 07/044,796 and WO 98/41511; European Patent Application EP 1916240-A and Piatak et al., Journal of Medicinal Chemistry 1964, 7(5), 590-592. Also, Example 1, Step C and Example 2, Step C illustrate the preparation of a compound of Formula 5.

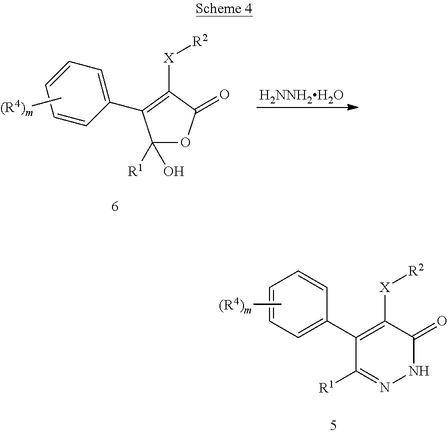

Alternatively, compounds of Formula 5 wherein R¹ is other than H can be prepared from compounds of Formula 5a (Formula 5 wherein R¹ is H, prepared by the method of Scheme 4) as outlined in Scheme 5. In the first step, the amide nitrogen in the compound of Formula 5a is protected, followed by halogenation to provide the intermediate of Formula 5b. Nitrogen-protecting groups and methods for protecting nitrogen atoms with these protecting groups are described in Greene, T. W.; Wuts, P. G. M. Protective Groups in Organic Synthesis, 2nd ed.; Wiley: New York, 1991. Halogenation can be done using methods analogous to those already described for Scheme 3. The protecting group on Formula 5b can be removed by standard deprotection conditions to give compounds of Formula 5 wherein R¹ is halogen. Also, compounds of Formula 5b can be subjected to various nucleophilic and metallation coupling reactions (using methods analogous to those already described for Scheme 1) to provide compounds of Formula 5 wherein R¹ is other than halogen. For example, compounds of Formula 5 wherein R¹ is alkyl, alkenyl, alkynyl, or the like can be synthesized from compounds of Formula 5b using various Grignard reagents in conjunction with a nickel catalyst. The general method of Scheme 5 is described PCT Patent Application Publication WO 09/086,041.

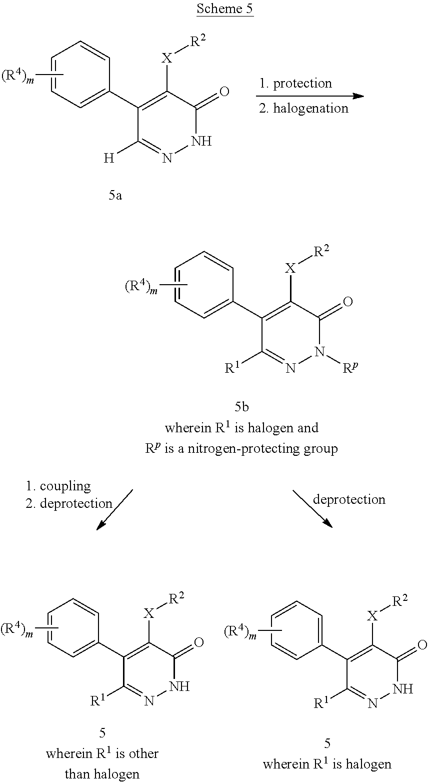

Compounds of Formula 6 can be synthesized by oxidation of furanones of Formula 7 as shown in Scheme 6. The oxidation reaction can be performed by contacting a compound of Formula 7 with an oxygen-containing gas such as air or oxygen, for example by bubbling oxygen or air into a reaction mixture comprising a compound of Formula 7. The reaction is conducted in a suitable solvent such as acetonitrile, ethyl acetate or tetrahydrofuran and optionally in the presence of a catalyst such as activated charcoal or a transition metal such as those comprising palladium, copper or iron. General procedures for conducting oxidations using an oxygen-containing gas are known in the art; see, for example, PCT Patent Application Publications WO 08/049,585 and WO 96/36623 and Nicoll-Griffith et al., Bioorganic and Medicinal Chemistry Letters 2000, 10, 2683-2686. Oxidation of Formula 7 using more potent oxidizers such as 3-chloroperbenzoic acid (MCPBA) in a solvent such chloroform can also be used.

Alternatively, compounds of Formula 6 can be chlorinated or brominated by treatment with N-chlorosuccinimide (NCS) or N-chlorosuccinimide (NBS) to give intermediates of Formula 8. The intermediates of Formula 8 can subsequently be hydrolyzed to provide compounds of Formula 6 using a catalytic amount of an acid such as acetic acid in a solvent system such as tetrahydrofuran and water according to the procedure given by Li et al., Bioorganic Medicinal Chemistry Letters 1976, 21, 1839-1842 and the procedure disclosed in PCT Patent Application Publication WO 98/41511. In view of the simplicity of operation, low cost of reactants and ease of isolating the desired product, the contact oxidation method using an oxygen-containing gas is most advantageous.

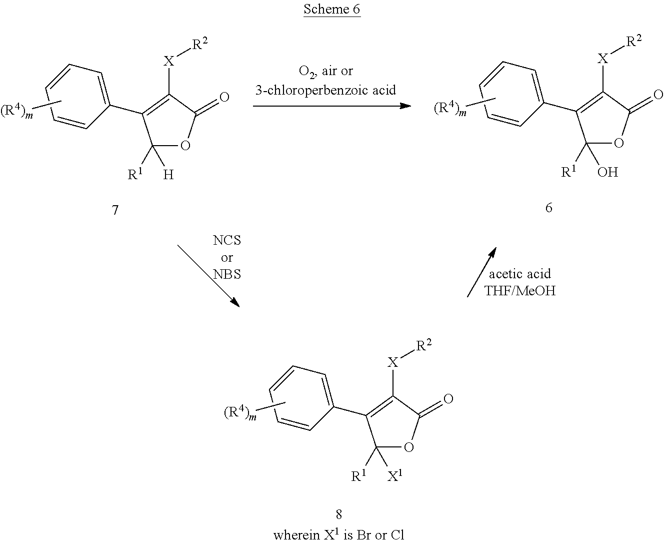

As shown in Scheme 7, the preparation of a compound of Formula 7 can be accomplished by reacting an α-haloketone of Formula 9 with an acetic acid of Formula 10 in the presence of a suitable base (e.g., a tertiary amine base such as triethylamine or an inorganic base such sodium hydroxide or potassium carbonate) to provide the corresponding ester, which undergoes intramolecular cyclization in the presence of 1,8-diazabicyclo[5.4.0]undec-7-ene (DBU) to form a compound of Formula 7. In practice the cyclization and oxidation (i.e. oxidation method of Scheme 6) can be done sequentially in one reaction vessel. Typical reaction conditions involve combining the compounds of Formulae 9 and 10 and the base in a solvent such as methanol, dioxane, tetrahydrofuran, acetonitrile, dimethylsulfoxide or N,N-dimethylformamide at a temperature between about 5 and 25° C. Preferably the reaction is run using an excess of the base relative to the compounds of Formulae 9 and 10, usually in the range of about 1.5 to about 3 molar equivalents. After formation of the ester (about 8 to 24 h), the reaction mixture is treated with DBU to promote cyclization, followed by passing a stream of air or oxygen through the reaction mixture. For a further description of the method of Scheme 7, see European Patent Application EP 1916240-A; Black et al., Bioorganic and Medicinal Chemistry Letters 2003, 13, 1195-1198 and Padakanti et al., Tetrahedron Letters 2002, 43, 8715-8719. Also, Example 1, Step B and Example 2, Step B illustrates the method of Scheme 7 where the cyclization and oxidation steps are done sequentially without isolation of a compound of Formula 7.

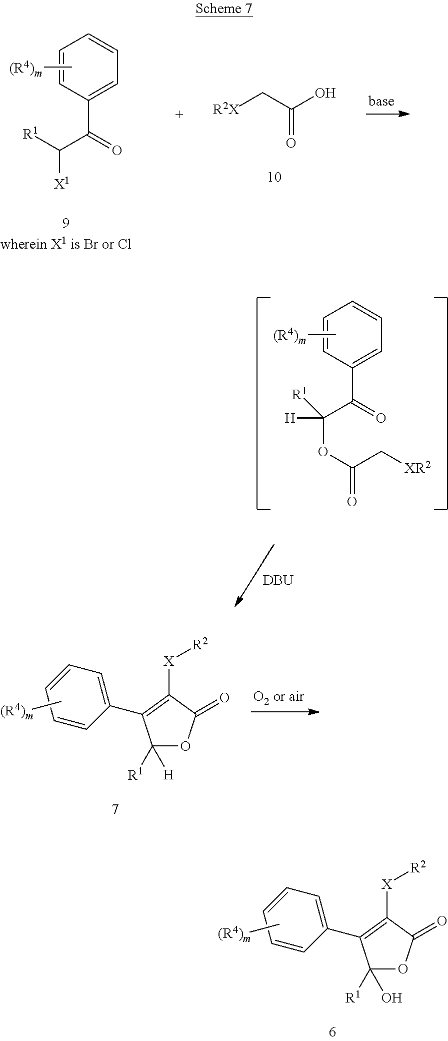

Compounds of Formula 9 are commercially available and can also be prepared from the corresponding ketones by standard halogenation methods known in the art. Particularly useful halogenating reagents for preparing compounds of Formula 9 include elemental halogen (Cl₂, Br₂), N-halosuccinimides (NBS, NCS), copper(II) halides (e.g., CuBr₂, CuCl₂) and pyridinium bromide perbromide. Example 1, Step A and Example 2, Step A illustrate the preparation of a compound of Formula 9.

In a method analogous to Scheme 7, an α-haloketone of Formula 11 can be reacted with a phenyl acetic acid of Formula 12 to form a compound of Formula 13 as shown in Scheme 8. Compounds of Formula 13 can subsequently be converted to Formula 1 using methods analogous to those described for Schemes 4 and 3. Example 6, Step C illustrates the method of Scheme 8.

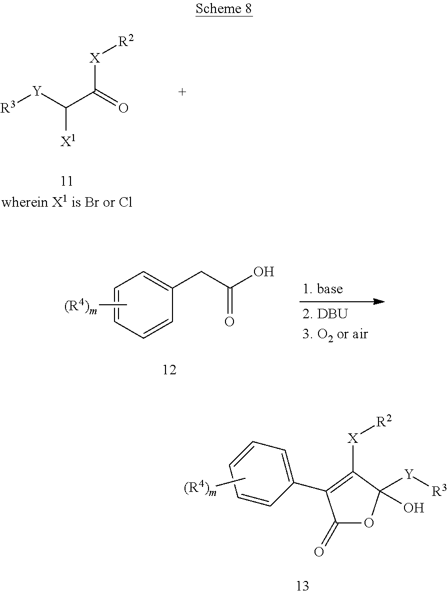

Alternatively, compounds of Formula 13 can be prepared by the four-step synthesis outlined in Scheme 9. In the first step, the dibromo ester of Formula 15 is prepared by reacting a phenylglyoxylate of Formula 14 with 2 equivalents of carbon tetrabromide in the presence of triphenylphosphine in a solvent such as chloroform of dichloromethane. Subsequent treatment of Formula 15 with a Grignard reagent, followed by reaction with an electrophile of formula R³Y—CHO provides compounds of Formula 16. For typical reaction conditions see Knochel et al., Synthesis 2003, 12, 1797-1802. Also, Example 14, Steps C and D illustrated the preparation of a compound of Formula 17.

Metal-catalyzed Suzuki coupling reactions can then be performed to introduce the R²X substituent onto the pyridazine ring, thus providing a compound of Formula 17. Oxidation of furanones Formula 17 can be done using a method analogous to Scheme 6 to provide compounds of Formula 13.

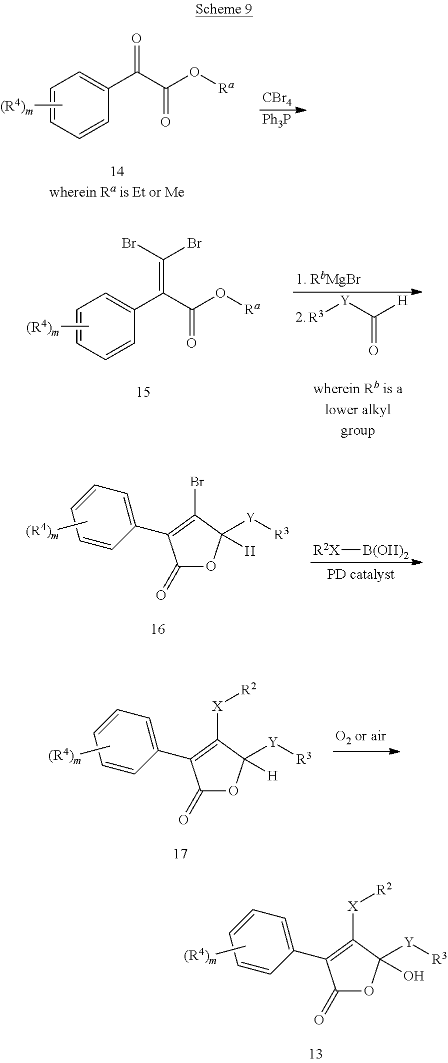

One skilled in the art will recognize that for some compounds of Formula 1, the R⁴ substituent(s) attached to the phenyl ring and the R⁵ and R⁶ substituents attached to the rings of R² and R³ may be more conveniently incorporated after forming the central pyridazine ring with the phenyl, R² and R³ rings attached. In particular, when R⁴, R⁵ and/or R⁶ is halogen or another suitable leaving group, the leaving group can be replaced using various electrophilic, nucleophilic and organometallic reactions known in the art to introduce other functional groups as R⁴, R⁵ and/or R⁶. Example 8 demonstrates the preparation of a compound of Formula 1 wherein R⁴ is methoxy starting from the corresponding compound of Formula 1 wherein R⁴ is fluoro. Example 10 illustrates the preparation of a compound of Formula 1 wherein R⁵ is chloro starting from the corresponding compound of Formula 1 wherein R⁵ is hydrogen. Example 13 illustrates the preparation of a compound of Formula 1 wherein R⁴ is trimethylsilyl (Me₃ Si—) starting from the corresponding compound of Formula 1 wherein R⁴ is hydrogen.

Furthermore, compounds of Formula 1 wherein the R⁴, R⁵ and/or R⁶ is —Z—V—W (as defined in the Summary of the Invention) can be prepared from the corresponding compounds of Formula 1 wherein R⁴, R⁵ and/or R⁶ is halogen or another suitable leaving group, such as by the general method described in PCT Patent Publication WO 2007/149448 (see Scheme 15 therein). This reference also describes other general methods for forming an R⁴, R⁵ and/or R⁶ substituent as —Z—V—W (see Schemes 16-19 therein). Present Example 9 demonstrates the preparation of a compound of Formula 1 wherein R⁴ is —Z—V—W (i.e. —O(CH₂)₃NMe₂) starting from the corresponding compound of Formula 1 wherein R⁴ is fluoro.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “m” means multiplet, “q” means quartet, “td” means triplet of doublets.

### Example 1

### Preparation of 4-(3,5-dimethoxyphenyl)-5-(4-fluorophenyl)-6-methyl-3-phenylpyridazine (Compound 1)

### Step A: Preparation of 2-bromo-1-(4-fluorophenyl)-1-propanone

To a mixture of 1-(4-fluorophenyl)-1-propanone (10.1 g, 66 mmol) in acetic acid (80 mL) was added bromine (3.3 mL, 64.4 mmol) dropwise followed by 3 drops of hydrobromic acid (48% in water). After 1 h, the reaction mixture was concentrated under reduced pressure to provide the title compound as a light orange oil (14.6 g).

¹H NMR (CDCl₃): δ 8.1 (m, 2H), 7.15 (m, 2H), 5.25 (m, 1H), 1.9 (d, 3H).

### Step B: Preparation of 3-(3,5-dimethoxyphenyl)-4-(4-fluorophenyl)-5-hydroxy-5-methyl-2(5H)-furanone

To a mixture of 2-bromo-1-(4-fluorophenyl)-1-propanone (i.e. the product of Step A) (5.89 g, 25.5 mmol) and 3,5-dimethoxybenzeneacetic acid (5.0 g, 25.5 mmol) in acetonitrile (170 mL) was added triethylamine (7.81 mL, 56.1 mmol). The reaction mixture was stirred overnight, and then 1,8-diazabicyclo[5.4.0]undec-7-ene (DBU) (6.53 mL, 43.3 mmol) was added. After 45 minutes, air was bubbled below the surface of the reaction mixture for 4 h. The reaction mixture was diluted with hydrochloric acid (1 N) and ethyl acetate, the layers were separated, and the aqueous layer was extracted with ethyl acetate. The combined organic layers were washed with saturated aqueous sodium chloride solution, dried over magnesium sulfate, filtered and concentrated under reduced pressure. The resulting material was purification by medium pressure liquid column chromatography (80 g of silica gel, 5 to 30% gradient ethyl acetate in hexanes as eluant) to provided the title compound as an oil (9.7 g).

¹H NMR (CDCl₃): δ 8.1 (m, 2H), 7.15 (m, 2H), 5.25 (m, 1H), 1.9 (d, 3H).

### Step C: Preparation of 4-(3,5-dimethoxyphenyl)-5-(4-fluorophenyl)-6-methyl-3(2H)-pyridazinone

To a mixture of 3-(3,5-dimethoxyphenyl)-4-(4-fluorophenyl)-5-hydroxy-5-methyl-2(5H)-furanone (i.e. the product of Step B) (7.47 g, 21.7 mmol) in n-butanol (43 mL) was added hydrazine monohydrate (2.74 mL, 56.4 mmol). The reaction mixture was heated at reflux for 2 h and then allowed to cool to room temperature. The reaction mixture was concentrated under reduced pressure, diluted with toluene and again concentrate. Diethyl ether and hexanes were added to the resulting solid and the mixture was filtered to provide the title compound as a white solid (4.8 g).

¹H NMR (CDCl₃): δ 7.4 (m, 1H), 7.0 (m, 3H), 6.5 (s, 1H), 6.4 (s, 1H), 6.3 (s, 1H), 6.2 (s, 1H), 3.66 (s, 3H), 3.63 (s, 3H), 2.10 (s, 3H).

### Step D: Preparation of 3-chloro-4-(3,5-dimethoxyphenyl)-5-(4-fluorophenyl)-6-methylpyridazine

A mixture of 4-(3,5-dimethoxyphenyl)-5-(4-fluorophenyl)-6-methyl-3(2H)-pyridazinone (i.e. the product of Step C) (4.7 g, 13.8 mmol) and phosphorus oxychloride (40 mL) was heated at reflux for 1 h. The reaction mixture was concentrated under reduced pressure, diluted with toluene and again concentrated. The resulting material was partitioned between dichloromethane and saturated aqueous sodium bicarbonate solution, the layers were separated, and the aqueous layer was extracted with dichloromethane (2×). The combined organic layers were washed with saturated aqueous sodium chloride solution, dried over magnesium sulfate, filtered and concentrated under reduced pressure. The resulting material was purified by medium pressure liquid column chromatography (40 g of silica gel, 5 to 30% gradient ethyl acetate in hexanes as eluant) to provided the title compound as a solid (1.64 g).

¹H NMR (CDCl₃): δ 7.0 (d, 4H), 6.34 (s, 1H), 6.15 (s, 2H), 3.67 (s, 6H), 2.51 (s, 3H).

### Step E: Preparation of 4-(3,5-dimethoxyphenyl)-5-(4-fluorophenyl)-6-methyl-3-phenylpyridazine

To a mixture of 3-chloro-4-(3,5-dimethoxyphenyl)-5-(4-fluorophenyl)-6-methylpyridazine (i.e. the product of Step D) (0.30 g, 0.84 mmol) and phenylboronic acid (0.153 g, 1.25 mmol) in p-dioxane (8.4 mL) was added tris(dibenzylideneacetone)-dipalladium(0) (30 mg, 0.033 mmol), 2-dicyclohexylphosphino-2′,6′-dimethoxybiphenyl (27 mg, 0.067 mmol) and potassium phosphate (ground just prior to use) (0.44 g, 2.1 mmol). The reaction mixture was heated at reflux overnight, then cooled to room temperature and diluted with water and ethyl acetate. The water/ethyl acetate mixture was filtered through a bed of Celite® (diatomaceous filter aid) in a sintered glass frit funnel and the Celite® was rinsed with water and ethyl acetate. The water/ethyl acetate filtrate was separated and the aqueous layer was extracted with ethyl acetate. The combined organic layers were washed with saturated aqueous sodium chloride solution, dried over magnesium sulfate, filtered and concentrated under reduced pressure. The resulting material was purified by flash column chromatography using a Bond Elute® tube (manufactured by Varian) prepacked with 10 g of silica gel (50 μm particle diameter, 70 Å pore size) (40% ethyl acetate in hexanes as eluent) to provide an oil (0.35 g). The oil was triturated with diethyl ether and hexanes to provide the title compound, a compound of the present invention, as a solid (239 mg) melting at 172-174° C.

¹H NMR (CDCl₃): δ 7.3 (2H), 7.2 (3H), 7.0 (4H), 6.18 (s, 1H), 5.9 (s, 2H), 3.49 (s, 6H), 2.58 (s, 3H).

### Example 2

### Preparation of 4-(3,5-dimethoxyphenyl)-3-(2-fluorophenyl)-5-(4-methoxyphenyl)-6-methylpyridazine (Compound 8)

### Step A: Preparation of 2-bromo-1-(4-methoxyphenyl)-1-propanone

To a mixture of 1-(4-methoxyphenyl)-1-propanone (15.0 g, 91 mmol) in dichloromethane (210 mL) was added pyridinium bromide perbromide (325 g, 91.3 mmol). The reaction mixture was stirred for 12 h, then diluted with water, and the layers were separated. The aqueous layer was extracted with dichloromethane, and the combined organic layers were washed with saturated aqueous sodium bisulfite solution and saturated aqueous sodium chloride solution, dried over magnesium sulfate, filtered and concentrated under reduced pressure to provide the title compound as a solid (23 g).

¹H NMR (CDCl₃): δ 8.0 (d, 2H), 6.9 (d, 2H), 5.2 (m, 1H), 3.88 (s, 3H), 1.8 (d, 3H).

### Step B: Preparation of 3-(3,5-dimethoxyphenyl)-5-hydroxy-4-(4-methoxyphenyl)-5-methyl-2(5H)-furanone

To a mixture of 2-bromo-1-(4-methoxyphenyl)-1-propanone (i.e. the product of Step A) (6.2 g, 25.5 mmol) and 3,5-dimethoxybenzeneacetic acid (5.0 g, 25.5 mmol) in acetonitrile (170 mL) was added triethylamine (7.81 mL, 56.1 mmol). The reaction mixture was stirred for 12 h, and then DBU (6.53 mL, 43.3 mmol) was added. After 1 h, air was bubbled below the surface of the reaction mixture for 4 h. The reaction mixture was diluted with hydrochloric acid (1 N) and ethyl acetate, the layers were separated, and the aqueous layer was extracted with ethyl acetate. The combined organic layers were washed with saturated aqueous sodium chloride solution, dried over magnesium sulfate, filtered and concentrated under reduced pressure. Diethyl ether and hexanes were added to the resulting solid and the mixture was filtered to provide the title compound as a solid (6.67 g).

¹H NMR (CDCl₃): δ 7.5 (d, 2H), 6.8 (d, 2H), 6.5 (s, 2H), 6.4 (s, 1H), 3.82 (s, 3H), 3.7 (s, 6H), 1.74 (s, 3H).

### Step C: Preparation of 4-(3,5-dimethoxyphenyl)-5-(4-methoxyphenyl)-6-methyl-3(2H)-pyridazine

To a mixture of 3-(3,5-dimethoxyphenyl)-5-hydroxy-4-(4-methoxyphenyl)-5-methyl-2(5H)-furanone (i.e. the product of Step B) (6.67 g, 18.7 mmol) in n-butanol (37 mL) was added hydrazine monohydrate (2.27 mL, 46.8 mmol). The reaction mixture was heated at reflux for 2 h and then allowed to cool to room temperature. The reaction mixture was concentrated under reduced pressure, diluted with toluene and again concentrated to provide the title compound as a solid (7 g).

¹H NMR (CDCl₃): δ 6.9 (d, 2H), 6.8 (d, 2H), 6.5 (s, 1H), 6.26 (s, 2H), 3.7 (s, 3H), 3.66 (s, 3H), 3.63 (s, 3H), 2.11 (s, 3H).

### Step D: Preparation of 3-chloro-4-(3,5-dimethoxyphenyl)-5-(4-methoxyphenyl)-6-methylpyridazine

A mixture of 4-(3,5-dimethoxyphenyl)-5-(4-methoxyphenyl)-6-methyl-3(2H)-pyridazine (i.e. the product of Step C) (6.59 g, 18.7 mmol) and phosphorus oxychloride (50 mL) was heated at reflux for 2 h. The reaction mixture was concentrated under reduced pressure, diluted with toluene and again concentrated. The resulting material was partitioned between dichloromethane and saturated aqueous sodium bicarbonate solution, the layers were separated, and the aqueous layer was extracted with dichloromethane. The combined organic layers were washed with saturated aqueous sodium bicarbonate solution and saturated aqueous sodium chloride solution, dried over magnesium sulfate, filtered and concentrated under reduced pressure. Diethyl ether and hexanes were added to the resulting solid and the mixture was filtered to provide the title compound as a solid (4.77 g).

¹H NMR (CDCl₃): δ 6.9 (d, 2H), 6.8 (d, 2H), 6.3 (s, 1H), 6.17 (s, 2H), 3.78 (s, 3H), 3.67 (s, 3H), 2.52 (s, 3H).

### Step E: Preparation of 4-(3,5-dimethoxyphenyl)-3-(2-fluorophenyl)-5-(4-methoxyphenyl)-6-methylpyridazine

To a mixture of 3-chloro-4-(3,5-dimethoxyphenyl)-5-(4-methoxyphenyl)-6-methylpyridazine (i.e. the title product of Step D) (0.3 g, 0.9 mmol) and 2-fluorophenylboronic acid (0.18 g, 1.3 mmol) in p-dioxane (8.8 mL) was added tris(dibenzylideneacetone)dipalladium(0) (32 mg, 0.035 mmol), 2-dicyclohexylphosphino-2′,6′-dimethoxybiphenyl (29 mg, 0.07 mmol) and potassium phosphate (ground just prior to use) (0.47 g, 2.2 mmol). The reaction mixture was heated at reflux overnight, then cooled to room temperature and diluted with water and ethyl acetate. The layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by flash column chromatography using a Bond Elute® tube (manufactured by Varian) prepacked with 10 g of silica gel (50 μm particle diameter, 70 Å pore size) (20% to 40% gradient of ethyl acetate in hexanes as eluent) to provide the title compound, a compound of the present invention, as a solid (145 mg)

¹H NMR (CDCl₃): δ 7.4 (t, 1H), 7.3 (m, 1H), 7.1 (t, 1H), 6.97 (d, 2H), 6.9 (t, 1H), 6.83 (d, 2H), 6.1 (s, 1H), 5.96 (s, 2H), 3.79 (s, 3H), 3.47 (s, 6H), 2.62 (s, 3H).

### Example 3

### Preparation of 4-(3,5-dimethoxyphenyl)-5-(4-fluorophenyl)-6-methyl-3-phenylpyridazine 1-oxide (Compound 12)

To a mixture of 4-(3,5-dimethoxyphenyl)-3-(2-fluorophenyl)-5-(4-methoxyphenyl)-6-methylpyridazine (i.e. the product of Example 1, Step E) (100 mg, 0.25 mmol) in dichloromethane (5 mL) was added 3-chlorobenzenecarboperoxoic acid (MCPBA) (77%, 56 mg, 0.25 mmol). The reaction mixture was stirred at room temperature overnight, then diluted with saturated aqueous sodium bisulfite solution and dichloromethane. The layers were separated, and the aqueous layer was extracted with dichloromethane. The combined organic layers were washed with saturated aqueous sodium bicarbonate solution (2×) and saturated aqueous sodium chloride solution, dried over magnesium sulfate, filtered and concentrated under reduced pressure to provide the title compound, a compound of the present invention, as a yellow solid (100 mg).

¹H NMR (CDCl₃): δ 7.37 (d, 2H), 7.28-7.2 (m, 3H), 7.0 (m, 4H), 6.18 (s, 1H), 5.9 (s, 2H), 3.49 (s, 6H), 2.4 (s, 3H).

### Example 4

### Preparation of 4-(3,5-dimethoxyphenyl)-6-methyl-5-phenyl-3-(2-pyridinyl)pyridazine (Compound 13)

A mixture of 3-chloro-4-(3,5-dimethoxyphenyl)-6-methyl-5-phenylpyridazine (prepared from 4-(3,5-dimethoxyphenyl)-6-methyl-5-phenyl-3(2H)-pyridazinone analogous to the procedure of Example 1) (0.3 g, 0.88 mmol), 2-(trimethylstannyl)pyridine (0.22 g, 0.88 mmol) and dichlorobis(triphenylphosphine)palladium (31 mg, 0.044 mmol) in N,N-dimethylformamide (8 mL) was heated at 85° C. overnight, and then at 100° C. for 4 h. The reaction mixture was cooled to room temperature, diluted with water and diethyl ether, and then filtered through a bed of Celite® (diatomaceous filter aid) in a sintered glass frit funnel and the Celite® was rinsed with water and diethyl ether. The water/diethyl ether filtrate was separated and the aqueous layer extracted with diethyl ether (2×). The combined organic layers were washed with aqueous cesium fluoride solution, water (3×) and saturated aqueous sodium chloride solution, dried over sodium sulfate, filtered and concentrated under reduced pressure. The resulting oil was purified by flash column chromatography using a Bond Elute® tube (manufactured by Varian) prepacked with 10 g of silica gel (50 μm particle diameter, 70 Å pore size) (30% to 40% gradient of ethyl acetate in hexanes as eluent) to provide the title compound, a compound of the present invention, as a solid (90 mg)

¹H NMR (CDCl₃): δ 8.6 (d, 1H), 8.4 (d, 1H), 7.8 (t, 1H), 7.28 (m, 1H), 7.2 (m, 3H), 7.0 (m, 2H), 6.3 (s, 1H), 6.17 (s, 2H), 3.65 (s, 6H), 2.51 (s, 3H).

### Example 5

### Preparation of 3-(chloromethyl)-5-(3,5-dimethoxyphenyl)-4-(4-fluorophenyl)-6-phenylpyridazine (Compound 14)

To a mixture of 4-(3,5-dimethoxyphenyl)-5-(4-fluorophenyl)-6-methyl-3-phenylpyridazine 1-oxide (i.e. the product of Example 3) (100 mg, 0.24 mmol) was added phosphorus oxychloride (6 mL). The reaction mixture was heated at reflux for 2 h, concentrated under reduced pressure, diluted with toluene and again concentrated. The resulting material was partitioned between dichloromethane and saturated aqueous sodium bicarbonate solution, the layers were separated, and the aqueous layer was extracted with dichloromethane. The combined organic layers were washed with saturated aqueous sodium chloride solution, dried over magnesium sulfate, filtered and concentrated under reduced pressure to provide the title compound, a compound of the present invention, as a solid (90 mg).

¹H NMR (CDCl₃): δ 7.4 (d, 2H), 7.2 (m, 3H), 7.18 (m, 2H), 7.0 (m, 2H), 6.2 (s, 1H), 5.9 (s, 2H), 4.7 (s, 2H), 3.5 (s, 6H).

### Example 6

### Preparation of 3-chloro-5-(3,5-dimethoxyphenyl)-6-(2-fluorophenyl)-4-(2,4,6-trifluorophenyl)pyridazine (Compound 45)

### Step A: Preparation of 1-(3,5-dimethoxyphenyl)-2-(2-fluorophenyl)ethanone

To a mixture of magnesium turnings (5.7 g, 0.2 mol), iodine (catalytic amount) and 1,2-dibromoethane (2 drops) in diethyl ether (150 mL) at reflux was added dropwise a solution of 1-(bromomethyl)-2-fluorobenzene (31.45 g, 0.17 mol) in diethyl ether (140 mL) over 105 minutes. The reaction mixture was cooled 10° C., and a solution of 3,5-dimethoxybenzonitrile (22.51 g, 0.14 mol) in diethyl ether (130 mL) and tetrahydrofuran (80 mL) was added dropwise. The reaction mixture was heated at reflux for 5 h and then allowed to stand at room temperature overnight. The reaction mixture was diluted with hydrochloric acid (1 N, 300 mL), water (75 mL), ethyl acetate (500 mL) and more hydrochloric acid (1 N, 200 mL). After stirring for 1 h, the layers were separated, and the aqueous layer was extracted with ethyl acetate (2×). The combined organic layers were washed with saturated aqueous sodium chloride solution, dried over sodium sulfate, filtered and concentrated under reduced pressure. The resulting solid was triturated with hexanes to provide the title compound as a solid (31.3 g).

¹H NMR (CDCl₃): δ 7.4-7.3 (m, 2H), 7.17 (s, 2H), 7.17-7.10 (m, 2H), 6.66 (s, 1H), 4.28 (s, 2H), 3.83 (s, 3H), 3.80 (s, 3H).

### Step B: Preparation of 2-bromo-1-(3,5-dimethoxyphenyl)-2-(2-fluorophenyl)ethanone

To a mixture of 1-(3,5-dimethoxyphenyl)-2-(2-fluorophenyl)ethanone (i.e. the product of Step A) (31.3 g, 0.11 mol) in chloroform (126 mL) was added copper(II) bromide (50.97 g, 0.23 mol) and ethyl acetate (126 mL). The reaction mixture was heated at reflux for 5 h, cooled to room temperature, and filtered through a bed of Celite® (diatomaceous filter aid) in a sintered glass frit funnel, and the Celite® was rinsed with hot ethyl acetate. The filtrate was washed with saturated aqueous sodium bicarbonate solution, dried over magnesium sulfate, filtered and concentrated under reduced pressure. The resulting solid was triturated with hexanes to provide the title compound as a solid (32.71 g).

¹H NMR (CDCl₃): δ 7.6 (t, 1H), 7.3 (m, 1H), 7.18 (m, 1H), 7.1 (s, 2H), 7.0 (m, 1H), 6.69 (s, 1H), 6.64 (s, 1H), 3.8 (s, 6H).

### Step C: Preparation of 4-(3,5-dimethoxyphenyl)-5-(2-fluorophenyl)-5-hydroxy-3-(2,4,6-trifluorophenyl)-2(5H)-furanone

To a mixture of 2-bromo-1-(3,5-dimethoxyphenyl)-2-(2-fluorophenyl)ethanone (i.e. the product of Step B) (90.81 g, 0.26 mol) and 2,4,6-trifluorobenzeneacetic acid (48.88 g, 0.26 mol) in acetonitrile (643 mL) was added triethylamine (60.95 mL, 0.44 mol). The reaction mixture was stirred for 3.5 h, and then DBU (85.26 mL, 0.57 mol) was added. After 1 h, air was bubbled below the surface of the reaction mixture for 1 h. The reaction mixture was diluted with hydrochloric acid (1 N), the layers were separated, and the aqueous layer was extracted with ethyl acetate (3×) The combined organic layers were washed with saturated aqueous sodium bicarbonate solution (3×), saturated aqueous sodium chloride solution, dried over magnesium sulfate and concentrated under reduced pressure. The resulting material was triturated with hexanes and ethyl acetate to provide the title compound as a white solid (6.32 g).

¹H NMR (CDCl₃): δ 7.8 (t, 1H), 7.3 (m, 1H), 7.1 (t, 1H), 7.0 (m, 1H), 6.78 (t, 1H), 6.70 (t, 1H), 6.4 (d, 2H), 6.3 (s, 1H), 4.38 (br s, 1H), 3.55 (s, 6H).

### Step D Preparation of 5-(3,5-dimethoxyphenyl)-6-(2-fluorophenyl)-4-(2,4,6-trifluorophenyl)-3(2H)-pyridazone

To a mixture of 4-(3,5-dimethoxyphenyl)-5-(2-fluorophenyl)-5-hydroxy-3-(2,4,6-trifluorophenyl)-2(5H)-furanone (i.e. the product of Step C) (35.99 g, 67.3 mmol) in ethanol (80 mL) was added hydrazine monohydrate (5 mL, 103 mmol). The reaction mixture was heated at reflux overnight, then cooled to room temperature, and filtered to provide the title compound as a white solid (22.71 g).

¹H NMR (CDCl₃): δ 11.8 (br s, 1H), 7.2 (m, 1H), 7.1 (t, 1H), 6.9 (t, 1H), 6.6 (m, 2H), 6.2 (s, 1H), 6.0 (s, 2H), 3.52 (s, 6H).

### Step E: Preparation of 3-chloro-5-(3,5-dimethoxyphenyl)-6-(2-fluorophenyl)-4-(2,4,6-trifluorophenyl)pyridazine

A mixture of 5-(3,5-dimethoxyphenyl)-6-(2-fluorophenyl)-4-(2,4,6-trifluorophenyl)-3(2H)-pyridazone (i.e. the product of Step D) (44.0 g, 96 mmol) and phosphorus oxychloride (200 mL) was heated at reflux for 1 h. The reaction mixture was cooled to room temperature, concentrated under reduced pressure, diluted with toluene and again concentrated (2×). The resulting material was partitioned between ethyl acetate and saturated aqueous sodium bicarbonate solution, the layers were separated, and the aqueous layer was extracted with ethyl acetate. The combined organic layers were washed with saturated aqueous sodium chloride solution, dried over magnesium sulfate, filtered and concentrated under reduced pressure. The resulting material was triturated with hexanes and diethyl ether to provide the title, a compound of the present invention, as a solid (41.91 g).

¹H NMR (CDCl₃): δ 7.4 (t, 1H), 7.3 (m, 1H), 7.1 (t, 1H), 6.9 (t, 1H), 6.6 (m, 2H), 6.23 (s, 1H), 6.0 (d, 2H), 3.53 (s, 6H).

### Example 7

### Preparation of 4-(3,5-dimethoxyphenyl)-3-(2-fluorophenyl)-6-methyl-5-(2,4,6-trifluorophenyl)pyridazine (Compound 24)

To a mixture of 3-chloro-5-(3,5-dimethoxyphenyl)-6-(2-fluorophenyl)-4-(2,4,6-trifluorophenyl)pyridazine (i.e. the product of Example 6, Step E) (41.9 g, 88.2 mmol) in p-dioxane (440 mL) was added dichloro[1,1′-bis(diphenylphosphino)ferrocene]palladium(II) dichloromethane complex (1:1) (7.2 g, 9 mmol), cesium carbonate (86.25 g, 264.7 mmol), 2,4,6-trimethylboroxine (11.08 g, 88.2 mol) and water (44 mL). The reaction mixture was heated at reflux for 1 h and then cooled to room temperature. The reaction mixture was partitioned between ethyl acetate and water, the layers were separated, and the aqueous layer was extracted with ethyl acetate. The combined organic layers were washed with saturated aqueous N,N′-1,2-ethanediylbis[N-(carboxymethyl)glycine (EDTA) solution and saturated aqueous sodium chloride solution, dried over magnesium sulfate, filtered and concentrated under reduced pressure. The resulting material was dissolved in ethyl acetate/hexanes and filtered through a bed of silica gel on a sintered glass frit funnel rinsed with ethyl acetate/hexanes (30%). The filtrate was concentrated under reduced pressure. The resulting solid was triturated with hexanes and diethyl ether and filtered to provide the title compound, a compound of the present invention, as a solid (28.91 g).

¹H NMR (CDCl₃): δ 7.4 (d, 2H), 7.3-7.2 (m, 2H), 7.1 (t, 1H), 6.9 (t, 1H), 6.96 (t, 2H), 6.2 (s, 1H), 6.0 (s, 2H), 3.53 (s, 6H), 2.61 (s, 3H).

### Example 8

### Preparation of 4-(2,6-difluoro-4-methoxyphenyl)-5-(3,5-dimethoxyphenyl)-6-(2-fluorophenyl)-3-methylpyridazine (Compound 28)

To a mixture of 4-(3,5-dimethoxyphenyl)-3-(2-fluorophenyl)-6-methyl-5-(2,4,6-trifluorophenyl)pyridazine (i.e. the product of Example 7) (200 mg, 0.44 mmol) in methanol (1.2 mL) was added sodium methoxide (25% solution, 15 mL, 0.7 mmol). The reaction mixture was heated at 60° C. overnight, then cooled, and diluted with water and ethyl acetate. The layers were separated, and the aqueous layer was extracted with ethyl acetate. The combined organic layers were washed with saturated aqueous sodium chloride solution, dried over magnesium sulfate, filtered and concentrated under reduced pressure. The resulting material was triturated with hexanes and diethyl ether to provide the title compound, a compound of the present invention, as a solid (144 mg).

¹H NMR (CDCl₃): δ 7.4 (m, 1H), 7.3 (m, 1H), 7.1 (m, 1H), 6.9 (m, 1H), 6.4 (d, 2H), 6.19 (s, H), 6.07 (s, 2H), 3.77 (s, 3H), 3.52 (s, 6H), 2.62 (s, 3H).

### Example 9

### Preparation of 3-[4-[5-(3,5-dimethoxyphenyl)-6-(2-fluorophenyl)-3-methyl-4-pyridazinyl]-3,5-difluorophenoxy]-N,N-dimethyl-1-propanamine (Compound 29)

To a mixture of 3-(dimethylamino)-1-propanol (72 mg, 0.70 mmol) in tetrahydrofuran (5 mL) was added sodium hydride (60% in mineral oil, 30 mg, 0.70 mmol). After stirring for 1 h, 4-(3,5-dimethoxyphenyl)-3-(2-fluorophenyl)-6-methyl-5-(2,4,6-trifluorophenyl)-pyridazine (i.e. the product of Example 7) (200 mg, 0.44 mmol) was added to the reaction mixture, and the mixture was heated at 60° C. overnight. The reaction mixture was cooled, diluted with water and ethyl acetate, and the layers were separated. The aqueous layer was extracted with ethyl acetate, and the combined organic layers were washed with saturated aqueous sodium chloride solution, dried over magnesium sulfate, filtered and concentrated under reduced pressure. The resulting material was purified by column chromatography (30% ethyl acetate in hexanes, and then methanol as eluant) to provide the title compound, a compound of the present invention, as an oil (100 mg).

¹H NMR (CDCl₃): δ 7.4 (m, 1H, 7.3 (m, 1H), 7.1 (m, 1H), 6.9 (m, 1H), 6.4 (d, 2H), 6.19 (s, H, 6.07 (s, 2H), 3.9 (t, 2H), 3.52 (s, 6H), 2.61 (s, 3H), 2.4 (t, 2H), 2.23 (s, 6H), 1.9 (m, 2H).

### Example 10

### Preparation of 4-(2-chloro-3,5-dimethoxyphenyl)-3-(2-fluorophenyl)-6-methyl-5-(2,4,6-trifluorophenyl)pyridazine (Compound 31)

To a mixture of 4-(3,5-dimethoxyphenyl)-3-(2-fluorophenyl)-6-methyl-5-(2,4,6-trifluorophenyl)pyridazine (i.e. the product of Example 7) (200 mg, 0.44 mmol) in carbon tetrachloride (5 mL) was added N-chlorosuccinimide (71 mg, 0.53 mmol) and 2,2′-(1,2-diazenediyl)bis[2-methylpropanenitrile] (AIBN) (catalytic amount). The reaction mixture was heated at 60° C. overnight, then cooled, and diluted with water and ethyl acetate. The layers were separated, and the aqueous layer was extracted with ethyl acetate. The combined organic layers were washed with saturated aqueous sodium chloride solution, dried over magnesium sulfate, filtered and concentrated under reduced pressure. The resulting material was purified by column chromatography (30% ethyl acetate in hexanes as eluant) to provide the title compound, a compound of the present invention, as a solid (100 mg).

¹H NMR (CDCl₃): δ 7.4 (t, 1H), 7.3 (m, 1H), 7.1 (t, 1H), 6.9 (t, 1H), 6.7-6.6 (m, 2H), 6.26 (s, 1H), 6.23 (s, 1H), 3.70 (s, 3H), 3.62 (s, 3H), 2.64 (s, 3H).

### Example 11

### Preparation of 4-(3,5-dimethoxyphenyl)-3-(2-fluorophenyl)-6-methoxy-5-(2,4,6-trifluorophenyl)pyridazine (Compound 26)

To a mixture of 3-chloro-5-(3,5-dimethoxyphenyl)-6-(2-fluorophenyl)-4-(2,4,6-trifluorophenyl)pyridazine (i.e. the product of Example 6, Step E) (100 mg, 0.21 mmol) in methanol (5 mL) was added sodium methoxide (5.4 M, 41 μL, 0.22 mmol). The reaction mixture was heated at reflux for 2 h, and then more sodium methoxide (5.4 M, 8 μL, 0.04 mmol) was added. After heating at reflux for an additional 3 h, the reaction mixture was cooled and concentrated under reduced pressure. The resulting material was purified by column chromatography (5 to 20% gradient of ethyl acetate in hexanes as eluant) to provide the title compound, a compound of the present invention, as a colorless oil (74 mg).

¹H NMR (CDCl₃): δ 7.40 (td, 1H), 7.29 (m, 1H), 7.13 (td, 1H), 6.92 (td, 1H), 6.62 (m, 2H), 6.21 (t, 1H), 6.05 (d, 2H), 3.52 (s, 6H).

### Example 12

### Preparation of 4-(3,5-dimethoxyphenyl)-3-(2-fluorophenyl)-5-(2,4,5-trifluorophenyl)pyridazine (Compound 27)

To a mixture of 3-chloro-5-(3,5-dimethoxyphenyl)-6-(2-fluorophenyl)-4-(2,4,6-trifluorophenyl)pyridazine (i.e. the product of Example 6, Step E) (0.39 g, 0.8 mmol) in ethanol (10 mL) and was added triethylamine (0.23 mL, 1.6 mmol) and palladium on carbon (50% water by weight, 10%, 40 mg, 0.038 mmol). The reaction vessel was evacuated and repressurized with nitrogen (3×) and then with hydrogen (2×). A balloon filled with hydrogen was then connected to the reaction flask, and the reaction mixture was stirred at room temperature for 1.5 h. The reaction mixture was filtered through a bed of Celite® (diatomaceous filter aid) on a sintered glass frit funnel, and the filtrate was concentrated under reduced pressure. The resulting material was dissolved in diethyl ether, washed with water (2×), dried over magnesium sulfate, filtered and concentrated under reduced pressure. The resulting solid was purified by column chromatography (10 to 25% gradient of ethyl acetate in hexanes as eluant) to provide the title compound, a compound of the present invention, as a white solid (0.26 g).

¹H NMR (CDCl₃): δ 7.18 (td, 1H), 6.96 (td, 1H), 6.68 (m, 2H), 6.25 (t, 1H), 6.06 (d, 2H), 4.20 (s, 3H), 3.52 (s, 6H).

### Example 13

### Preparation of 3-chloro-5-(3,5-dimethoxyphenyl)-6-(2-fluorophenyl)-4-[2,4,6-trifluoro-3-(trimethylsilyl)phenyl]pyridazine (Compound 38)

To a mixture of 3-chloro-5-(3,5-dimethoxyphenyl)-6-(2-fluorophenyl)-4-(2,4,6-trifluorophenyl)pyridazine (i.e. the product of Example 6, Step E) (144 mg, 0.30 mmol) in tetrahydrofuran (20 mL) at −70° C. was added lithium bis(trimethylsilyl)amide (1 M in tetrahydrofuran, 550 μL, 0.54 mmol). The reaction mixture was stirred at −70° C. for 1 h, and then ethyl formate (52 mL, 0.63 mmol) was added. The reaction mixture was allowed to slowly warm to room temperature and stirred overnight. Saturated aqueous ammonium chloride solution was added to the reaction mixture, and the aqueous mixture was extracted with diethyl ether. The organic layer was dried over magnesium sulfate, filtered and concentrated under reduced pressure. The resulting material was purified by column chromatography (5 to 15% gradient of ethyl acetate in hexanes as eluant) to provide the title compound, a compound of the present invention, as a solid (16 mg).

¹H NMR (CDCl₃): δ 7.43 (td, 1H), 7.34 (m, 1H), 7.16 (td, 1H), 6.95 (t, 1H), 6.58 (td, 1H), 6.22 (t, 1H), 6.03 (br s, 2H), 3.53 (s, 6H), 0.28 (s, 9H).

### Example 14

### Preparation of 3-chloro-6-(2-fluorophenyl)-5-(5-methoxy-3-pyridinyl)-4-(2,4,6-trifluorophenyl)pyridazine (Compound 44)

### Step A: Preparation of ethyl α-oxo-1H-imidazoleacetate

To a mixture of ethyl oxalyl chloride (75.75 g, 0.55 mol) in tetrahydrofuran (500 mL) at 0° C. was added a solution of pyrazole (76.6 g, 1.10 mol) in tetrahydrofuran (400 mL). When the addition was complete, more tetrahydrofuran (100 mL) was added to the reaction mixture. After stirring overnight, the reaction mixture was filtered, and the filtrate was concentrated under reduced pressure to provide the title compound as an oil (89.9 g)

¹H NMR (CDCl₃): δ 8.54 (s, 1H), 7.6 (s, 1H), 7.18 (s, 1H), 4.5 (q, 2H), 1.46 (t, 3H).

### Step B: Preparation of ethyl α-oxo-2,4,6-trifluorobenzeneacetate

To a mixture of 1,3,5-trifluorobenzene (11.0 g, 83.6 mol) in tetrahydrofuran (200 mL) at −78° C. was added n-butyllithium (2.5 M in hexanes, 35.2 mL, 87.8 mol). The reaction mixture was stirred at −78 to −60° C. for 1 h, and then the reaction mixture was added to a solution of ethyl α-oxo-1H-imidazoleacetate (i.e. the product of Step A) (45 g, 267 mol) and tetrahydrofuran (340 mL) while maintaining the reaction temperature below −60° C. The reaction mixture was slowly warmed to room temperature and stirred for 1 h, and then cooled to approximately 0° C. and diluted with saturated aqueous ammonium chloride solution and ethyl acetate. The layers were separated, and the aqueous layer was extracted with ethyl acetate. The combined organic layers were washed with saturated aqueous sodium chloride solution, dried over magnesium sulfate, filtered and concentrated under reduced pressure. The resulting oil was purified by column chromatography (5 to 30% gradient of ethyl acetate in hexanes as eluant) to provide the title compound as an oil (13.4 g).

¹H NMR (CDCl₃): δ 6.78 (t, 2H), 4.4 (q, 2H), 1.39 (t, 3H).

### Step C: Preparation of ethyl α-(dibromoethylene)-2,4,6-trifluorobenzeneacetate

To a mixture of triphenylphosphine (13.56 g, 51.7 mmol) in dichloromethane (20 mL) at 0° C. was added a solution of carbon tetrabromide (8.57 g, 25.8 mmol) in dichloromethane (16 mL). The reaction mixture was stirred for 30 minutes at 0° C., and then a solution of ethyl α-oxo-2,4,6-trifluorobenzeneacetate (i.e. the product of Step B) (3 g, 12.9 mmol) in dichloromethane (8 mL) was added, and the mixture was allowed to warm to room temperature and stirred overnight. The reaction mixture was diluted with hexanes and filtered, and the filtrate was concentrated under reduced pressure. The resulting oil was purified by column chromatography (hexanes eluant) to provide the title compound as an oil (2.67 g).

¹H NMR (CDCl₃): δ 6.7 (t, 2H), 4.2 (q, 2H), 1.2 (t, 3H).

### Step D: Preparation of 4-bromo-5-(2-fluorophenyl)-3-(2,4,6-trifluorophenyl)-2(5H)-furanone

To a mixture of ethyl α-(dibromoethylene)-2,4,6-trifluorobenzeneacetate (i.e. the product of Step C) (4.52 g, 11.6 mmol) in diethyl ether (78 mL) at −78° C. was added isopropylmagnesium chloride (2 M in diethyl ether, 6.1 mL, 12.2 mmol). The reaction mixture was stirred for 3 h at −10 to 5° C., and then a solution of 2-fluorobenzaldehyde (1.47 mL, 13.9 mmol) in diethyl ether (3 mL) was added. After about 20 minutes, the reaction mixture was diluted with saturated aqueous sodium chloride solution and ethyl acetate. The layers were separated, and the aqueous layer was extracted with ethyl acetate. The combined organic layers were washed with saturated aqueous sodium chloride solution, dried over magnesium sulfate, filtered and concentrated under reduced pressure. The resulting solid was triturated with hexanes to provide the title as a solid (2.83 g).

¹H NMR (CDCl₃): δ 7.4 (m, 1H), 7.2 (m, 2H) 7.20 (t, 1H) 6.8 (t, 2H) 6.35 (s, 1H).

### Step E: Preparation of 5-(2-fluorophenyl)-4-(5-methoxy-3-pyridinyl)-3-(2,4,6-trifluorophenyl)-2(5H)-furanone

To a mixture of 4-bromo-5-(2-fluorophenyl)-3-(2,4,6-trifluorophenyl)-2(5H)-furanone (i.e. the product of Step D) (1.0 g, 2.7 mmol) in toluene (11 mL) was added 5-methoxypyridine-3-boronic acid (0.95 g, 4.0 mmol), dichlorobis(triphenylphosphine)-palladium (95 mg, 0.13 mmol), cesium fluoride (1.09 g, 7.2 mmol), N,N,N-triethylbenzenemethanaminium chloride (30 mg, 0.13 mmol) and water (11 mL). The reaction mixture was heated at reflux overnight, then cooled, and partitioned between ethyl acetate and water. The layers were separated, and the aqueous layer was extracted with ethyl acetate (2×). The combined organic layers were washed with saturated aqueous sodium chloride solution, dried over magnesium sulfate, filtered and concentrated under reduced pressure. The resulting oil was as purified by column chromatography (20 to 30% gradient of ethyl acetate in hexanes as eluant) to provide the title compound as an oil (1.3 g).

¹H NMR (CDCl₃): δ 8.2 (d, 1H), 8.0 (s, 1H), 7.3 (m, 1H), 7.2 (m, 1H), 7.1 (d, 1H), 7.0 (m, 1H), 6.9 (s, 1H), 6.8 (m, 1H), 6.78 (s, 1H), 6.7 (m, 1H), 3.71 (s, 3H).

### Step F: Preparation of 5-(2-fluorophenyl)-5-hydroxy-4-(5-methoxy-3-pyridinyl)-3-(2,4,6-trifluorophenyl)-2(5H)-furanone

To a solution of 5-(2-fluorophenyl)-4-(5-methoxy-3-pyridinyl)-3-(2,4,6-trifluorophenyl)-2(5H)-furanone (i.e. the product of Step E) (1.2 g, 2.9 mmol) in ethyl acetate (200 mL) was added Darco® G-60 (activated charcoal powder, −100 mesh particle size), and the reaction mixture was stirred under air overnight. The reaction mixture was filtered through a bed of Celite® (diatomaceous filter aid) on a sintered glass frit funnel, and the Celite® was rinsed with ethyl acetate. The filtrate was concentrated under reduced pressure to provide the title compound as a solid (0.84 g).

¹H NMR (CDCl₃): δ 8.1 (d, 1H), 8.0 (s, 1H), 7.8 (m, 1H), 7.3 (m, 1H), 7.2 (t, 1H), 7.1 (d, 1H), 7.0 (m, 1H), 6.7 (t, 2H), 3.6 (s, 3H).

### Step G: Preparation of 6-(2-fluorophenyl)-5-(5-methoxy-3-pyridinyl)-4-(2,4,6-trifluorophenyl)-3(2H)-pyridazone

To a mixture of 5-(2-fluorophenyl)-5-hydroxy-4-(5-methoxy-3-pyridinyl)-3-(2,4,6-trifluorophenyl)-2(5H)-furanone (i.e. the product of Step F) (0.84 g, 1.9 mmol) in ethanol (12 mL) was added hydrazine monohydrate (123 μL, 2.53 mmol). The reaction mixture was heated at reflux overnight. After cooling to room temperature, the reaction mixture was filtered to provide the title compound as a white solid (358 mg).

¹H NMR (CDCl₃): δ 8.0 (d, 1H), 7.6 (s, 1H), 7.5 (m, 1H), 7.4 (d, 1H), 7.22-7.20 (m, 3H), 7.0 (t, 1H), 6.9 (s, 1H), 3.5 (s, 3H).

### Step H: Preparation of 3-chloro-6-(2-fluorophenyl)-5-(5-methoxy-3-pyridinyl)-4-(2,4,6-trifluorophenyl)pyridazine

A mixture of 6-(2-fluorophenyl)-5-(5-methoxy-3-pyridinyl)-4-(2,4,6-trifluorophenyl)-3(2H)-pyridazone (i.e. the product of Step G) (358 mg, 0.84 mmol) and phosphorus oxychloride (4 mL) was heated at reflux for 90 minutes. The reaction mixture was concentrated under reduced pressure, diluted with toluene and again concentrated. The resulting material was partitioned between ethyl acetate and saturated aqueous sodium bicarbonate solution, the layers were separated, and the aqueous layer was extracted with ethyl acetate (2×). The combined organic layers were washed with saturated aqueous sodium chloride solution, dried over magnesium sulfate, filtered and concentrated under reduced pressure. The resulting material was purified by flash column chromatography (30% ethyl acetate in hexanes as eluant) to provide a solid. The solid was triturated with hexanes to provide the title compound, a compound of the present invention, as a solid (186 mg).

¹H NMR (CDCl₃): δ 8.1 (d, 1H), 7.7 (s, 1H), 7.3 (m, 1H), 7.2 (m, 1H), 6.9 (t, 1H), 6.7 (s, 1H), 6.68 (m, 2H), 3.61 (s, 3H).

By the procedures described herein together with methods known in the art, the following compounds of Tables 1 to 5 can be prepared. The following abbreviations are used in the Tables which follow: Me means methyl, Et means ethyl, MeO means methoxy, CN means cyano and Ph means phenyl.

TABLE 1

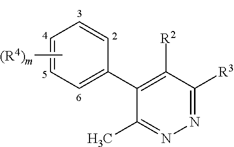

| R³ | R³ |
|---|---|
| R² is 3,5-di-MeO—Ph; (R⁴)ₘ is 2-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 2-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 2-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 4-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 4-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 4-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 2,4-di-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 2,4-di-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 2,4-di-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 2,6-di-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 2,6-di-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 2,6-di-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 2,4,6-tri-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 2,4,6-tri-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 2,4,6-tri-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 2,3,6-tri-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 2,3,6-tri-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 2,3,6-tri-F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 2-MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 2-MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 2-MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 4-MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R² is 2-Cl, 5-MeO—Ph; (R⁴)ₘ is 4-MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 2-Cl, 3,5-di-MeO—Ph; (R⁴)ₘ is 4-MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R² is 3,5-di-MeO—Ph; (R⁴)ₘ is 2-F. | |
| 1-Me-1H-5-pyrazolyl | tetrahydro-2H-pyran-4-yl |
| tetrahydro-2H-pyran-2-yl | tetrahydro-2H-pyran-3-yl |
| R² is 2-Cl, 3,5-di-MeO—Ph; (R⁴)ₘ is 2-F. | |
| 1-Me-1H-pyrazol-5-yl | tetrahydro-2H-pyran-4-yl |
| tetrahydro-2H-pyran-2-yl | tetrahydro-2H-pyran-3-yl |
| R² is 3,5-di-MeO—Ph; (R⁴)ₘ is 2,4-di-F. | |
| 1-Me-1H-5-pyrazolyl | tetrahydro-2H-pyran-4-yl |
| tetrahydro-2H-pyran-2-yl | tetrahydro-2H-pyran-3-yl |
| R² is 2-Cl, 3,5-di-MeO—Ph; (R⁴)ₘ is 2,4-di-F. | |
| 1-Me-1H-pyrazol-5-yl | tetrahydro-2H-pyran-4-yl |
| tetrahydro-2H-pyran-2-yl | tetrahydro-2H-pyran-3-yl |

TABLE 2

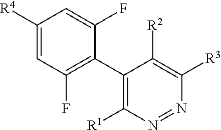

| R³ | R³ |
|---|---|
| R¹ is Cl; R² is 3,5-di-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R¹ is Cl; R² is 2-Cl, 5-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Cl; R² is 2-Cl, 3,5-di-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is CN; R² is 3,5-di-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R¹ is CN; R² is 2-Cl, 5-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is CN; R² is 2-Cl, 3,5-di-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Et; R² is 3,5-di-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R¹ is Et; R² is 2-Cl, 5-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Et; R² is 2-Cl, 3,5-di-MeO—Ph; R⁴ is F. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Cl; R² is 3,5-di-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R¹ is Cl; R² is 2-Cl, 5-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Cl; R² is 2-Cl, 3,5-di-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is CN; R² is 3,5-di-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R¹ is CN; R² is 2-Cl, 5-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is CN; R² is 2-Cl, 3,5-di-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Et; R² is 3,5-di-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 5-oxazoly |
| R¹ is Et; R² is 2-Cl, 5-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Et; R² is 2-Cl, 3,5-di-MeO—Ph; R⁴ is MeO. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 5-oxazoly |
| R¹ is Cl; R² is 3,5-di-MeO—Ph; R⁴ is F. | |
| 1-Me-1H-5-pyrazolyl | tetrahydro-2H-pyran-4-yl |
| tetrahydro-2H-pyran-2-yl | tetrahydro-2H-pyran-3 -yl |
| R¹ is Cl; R² is 2-Cl, 3,5-di-MeO—Ph; R⁴ is F. | |
| 1-Me-1H-pyrazol-5-yl | tetrahydro-2H-pyran-4-yl |
| tetrahydro-2H-pyran-2-yl | tetrahydro-2H-pyran-3 -yl |
| R¹ is Cl; R² is 3,5-di-MeO—Ph; R⁴ is MeO. | |
| 1-Me-1H-5-pyrazolyl | tetrahydro-2H-pyran-4-yl |
| tetrahydro-2H-pyran-2-yl | tetrahydro-2H-pyran-3-yl |
| R¹ is Cl; R² is 2-Cl, 3,5-di MeO—Ph; R⁴ is MeO. | |
| 1-Me-1H-pyrazol-5-yl | tetrahydro-2H-pyran-4-yl |
| tetrahydro-2H-pyran-2-yl | tetrahydro-2H-pyran-3-yl |

TABLE 3

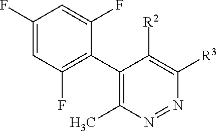

| R² | R³ |
|---|---|
| 3-MeO—Ph | Ph |
| 2-F, 5-MeO—Ph | Ph |
| 2-Br, 5-MeO—Ph | Ph |
| 2-F, 3,5-di-MeO—Ph | Ph |
| 2-Br, 3,5-di-MeO—Ph | Ph |
| 3-MeO—Ph | 4-F—Ph |
| 2-F, 5-MeO—Ph | 4-F—Ph |
| 2-Br, 5-MeO—Ph | 4-F—Ph |
| 2-F, 3,5-di-MeO—Ph | 4-F—Ph |
| 2-Br, 3,5-di-MeO—Ph | 4-F—Ph |
| 3-MeO—Ph | 2-F—Ph |
| 2-F, 5-MeO—Ph | 2-F—Ph |
| 2-Br, 5-MeO—Ph | 2-F—Ph |
| 2-F, 3,5-di-MeO—Ph | 2-F—Ph |
| 2-Br, 3,5-di-MeO—Ph | 2-F—Ph |

TABLE 4

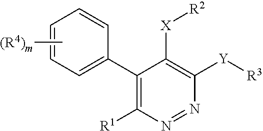

| R³ | R³ |
|---|---|
| R¹ is Me; R² is 3,5-di-MeO—Ph; (R⁴)ₘ is 2-F; | |
| X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Me; R² is 3,5-di-MeO—Ph; (R⁴)ₘ is 2-F; | |
| X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Me; R² is 2-Cl—Ph; (R⁴)ₘ is 2-F; X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R^{l} is Me; R² is 2-Cl—Ph; (R⁴)ₘ is 2-F; X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Me; R² is 3,5-di-MeO—Ph; (R⁴)ₘ is 4-F; | |
| X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Me; R² is 3,5-di-MeO—Ph; (R⁴)ₘ is 4-F; | |
| X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Me; R² is 2-Cl—Ph; (R⁴)ₘ is 4-F; X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Me; R² is 2-Cl—Ph; (R⁴)ₘ is 4-F; X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Cl; R² is 3,5-di-MeO—Ph; (R⁴)ₘ is 2-F; | |
| X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Cl; R² is 3,5-di-MeO—Ph; (R⁴)ₘ is 2-F; | |
| X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Cl; R² is 2-Cl—Ph; (R⁴)ₘ is 2-F; X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Cl; R² is 2-Cl—Ph; (R⁴)ₘ is 2-F; X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Cl; R² is 3,5-di-MeO—Ph; (R⁴)ₘ is 4-F; | |
| X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Cl; R² is 3,5-di-MeO—Ph; (R⁴)ₘ is 4-F; | |
| X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R¹ is Cl; R² is 2-Cl—Ph; (R⁴)ₘ is 4-F; X is CH₂; Y is a direct bond. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R¹ is Cl; R² is 2-Cl—Ph; (R⁴)ₘ is 4-F; X is a direct bond; Y is CH₂. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |

TABLE 5

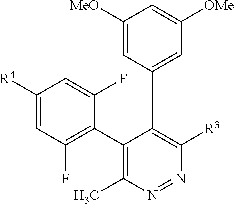

| R³ | R³ |
|---|---|
| R⁴ is 4-MeNH(CH₂)₃O. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R⁴ is 3-MeNH(CH₂)₃O. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R⁴ is 4-Me₂N(CH₂)₃O. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R⁴ is 3-Me₂N(CH₂)₃O. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |
| R⁴ is 4-MeO(CH₂)₃O. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-F—Ph | 1H-pyrazol-1-yl |
| 2,4-di-Cl—Ph | 1-Me-1H-5-pyrazolyl |
| R⁴ is 3-MeO(CH₂)₃O. | |
| Ph | 2-F, 4-Cl—Ph |
| 2-F—Ph | 2-F, 6-Cl—Ph |
| 2-Cl—Ph | 2-Cl, 4-F—Ph |
| 3-F—Ph | 2,3,6-tri-F—Ph |
| 3-Cl—Ph | 2,3,6-tri-Cl—Ph |
| 4-F—Ph | 2,4,6-tri-F—Ph |
| 4-Cl—Ph | 2,4,6-tri-Cl—Ph |
| 2-Me—Ph | 2-pyridinyl |
| 2-MeO—Ph | 3-pyridinyl |
| 4-MeO—Ph | 4-pyridinyl |
| 2-CF₃O—Ph | 5-Cl-2-pyridinyl |
| 4-CF₃O—Ph | 2-furanyl |
| 4-CN—Ph | 2-thienyl |
| 2,4-di-Cl—Ph | 1H-pyrazol-1-yl |
| 2,4-di-F—Ph | 1-Me-1H-5-pyrazolyl |

### FORMULATION/UTILITY

A compound of this invention will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylenes, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

Example A

| High Strength Concentrate | |
|---|---|
| Compound 13 | 98.5% |
| silica aerogel | 0.5% |
| synthetic amorphous fine silica | 1.0% |

Example B

| | |
|---|---|
| Wettable Powder | |
| Compound 17 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

Example C

| | |
|---|---|
| Granule | |
| Compound 30 | 10.0% |
| attapulgite granules (low volatile matter, 0.71/ | 90.0% |
| 0.30 mm; U.S.S. No. 25-50 sieves) | |

Example D

| | |
|---|---|
| Extruded Pellet | |
| Compound 33 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

Example E

| | |
|---|---|
| Emulsifiable Concentrate | |
| Compound 34 | 10.0% |
| polyoxyethylene sorbitol hexoleate | 20.0% |
| C₆—C₁₀ fatty acid methyl ester | 70.0% |

Example F

| | |
|---|---|
| Microemulsion | |
| Compound 13 | 5.0% |
| polyvinylpyrrolidone-vinyl acetate copolymer | 30.0% |
| alkylpolyglycoside | 30.0% |
| glyceryl monooleate | 15.0% |
| Water | 20.0% |

Example G

| | |
|---|---|
| Seed Treatment | |
| Compound 17 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds can be influenced by many factors of the environment and should be determined under actual use conditions. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸ →Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, N-[2-(1S,2R)-[1,1′-bicyclopropyl]-2-ylphenyl]-3-(difluoromethyl)-1-methyl-1H-pyrazole-4-carboxamide and N-[2-(1,3-dimethylbutyl)phenyl]-5-fluoro-1,3-dimethyl-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to f3-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorus acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-S-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)“, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7) and (46.4) “benzophenone fungicides” (Fungicide Resistance Action Committee (FRAC) code U8). The thiazole carboxamides include ethaboxam. The phenyl-acetamides include cyflufenamid and N-[[(cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrrolnitrin, quinomethionate, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl) [1,2,4]triazolo[1,5-c]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, amidoflumet (S-1955), avermectin, azadirachtin, azinphos-methyl, bifenthrin, bifenazate, 3-bromo-1-(3-chloro-2-pyridinyl)-N-[4-cyano-2-methyl-6-[(methylamino)-carbonyl]phenyl]-1H-pyrazole-5-carboxamide, buprofezin, carbofuran, cartap, chlorantraniliprole (DPX-E2Y45), chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) pyrimidinone fungicides; (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Pyrimidinone fungicides (group (4)) include compounds of Formula A1

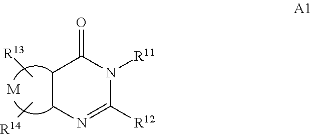

wherein M forms a fused phenyl, thiophene or pyridine ring; R¹¹ is C₁-C₆ alkyl; R¹² is C₁-C₆ alkyl or C₁-C₆ alkoxy; R¹³ is halogen; and R¹⁴ is hydrogen or halogen.

Pyrimidinone fungicides are described in PCT Patent Application Publication WO 94/26722 and U.S. Pat. Nos. 6,066,638, 6,245,770, 6,262,058 and 6,277,858. Of note are pyrimidinone fungicides selected from the group: 6-bromo-3-propyl-2-propyloxy-4(3H)-quinazolinone, 6,8-diiodo-3-propyl-2-propyloxy-4(3H)-quinazolinone, 6-iodo-3-propyl-2-propyloxy-4(3H)-quinazolinone (proquinazid), 6-chloro-2-propoxy-3-propyl-thieno[2,3-d]pyrimidin-4(3H)-one, 6-bromo-2-propoxy-3-propylthieno[2,3-d]pyrimidin-4(3H)-one, 7-bromo-2-propoxy-3-propylthieno[3,2-d]pyrimidin-4(3H)-one, 6-bromo-2-propoxy-3-propylpyrido[2,3-d]pyrimidin-4(3H)-one, 6,7-dibromo-2-propoxy-3-propyl-thieno[3,2-d]pyrimidin-4(3H)-one, and 3-(cyclopropylmethyl)-6-iodo-2-(propylthio)pyrido-[2,3-d]pyrimidin-4(3H)-one.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kresoxim-methyl, trifloxystrobin, pyraclostrobin, picoxystrobin, dimoxystrobin, metominostrobin/fenominostrobin, quinoxyfen, metrafenone, cyflufenamid, fenpropidine, fenpropimorph, cyproconazole, epoxiconazole, flusilazole, metconazole, propiconazole, proquinazid, prothioconazole, tebuconazole, triticonazole, famoxadone and penthiopyrad. Specifically preferred mixtures (compound numbers refer to compounds in Index Table A) are selected from the group: combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with azoxystrobin, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with kresoxim-methyl, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with trifloxystrobin, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with picoxystrobin, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with dimoxystrobin, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with metominostrobin/fenominostrobin, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with quinoxyfen, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with metrafenone, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with cyflufenamid, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with fenpropidine, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with fenpropimorph, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with cyproconazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with epoxiconazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with flusilazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with metconazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with propiconazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with proquinazid, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with prothioconazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with tebuconazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with triticonazole, combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with famoxadone, and combinations of Compound 2, Compound 7, Compound 9, Compound 15, Compound 18, Compound 24, Compound 25, Compound 26, Compound 28, Compound 30, Compound 31, Compound 35, Compound 36 or Compound 37 with penthiopyrad.

The rate of application required for effective control (i.e. “biologically effective amount”) will depend on such factors as the plant diseases to be controlled, the location, time of year, host crop, ambient moisture, temperature, and the like. One skilled in the art can easily determine through simple experimentation the biologically effective amount necessary for the desired level of plant disease control.

The following TESTS demonstrate the control efficacy of compounds of this invention on specific pathogens. The pathogen control protection afforded by the compounds is not limited, however, to these species. See Index Table A for compound descriptions. See Index Table B for ¹H NMR data. The following abbreviations are used in the Index Table: Me is methyl, MeO is methoxy and Ph is phenyl. The abbreviation “Cmpd.” stands for “Compound”, and the abbreviation “Ex.” stands for “Example” and is followed by a number indicating in which example the compound is prepared.

INDEX TABLE A

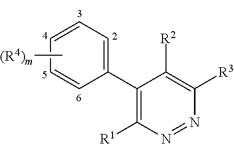

| Cmpd. | R¹ | (R⁴)ₘ | R² | R³ | m.p. (° C.) |
|---|---|---|---|---|---|
| 1 (Ex. 1) | Me | 4-F | 3,5-di-MeO—Ph | Ph | *** |
| 2 | Me | 4-F | 3,5-di-MeO—Ph | 2-F—Ph | ** |
| 3 | Me | - | 3,5-di-MeO—Ph | Ph | ** |
| 4 | Me | - | 3,5-di-MeO—Ph | 2-F—Ph | ** |
| 5 | Me | - | 3,5-di-MeO—Ph | 2,4-di-F—Ph | ** |
| 6 | Me | 4-Cl | 3,5-di-MeO—Ph | Ph | ** |
| 7 | Me | 4-MeO | 3,5-di-MeO—Ph | Ph | ** |
| 8 (Ex. 2) | Me | 4-MeO | 3,5-di-MeO—Ph | 2-F—Ph | *** |
| 9 | Me | 4-MeO | 3,5-di-MeO—Ph | 2,4-di-F—Ph | ** |
| 10 | Me | - | 3,5-di-MeO—Ph | 2,3-di-F—Ph | ** |
| 11 | Me | - | 3,5-di-MeO—Ph | 4-F—Ph | ** |
| 12 (Ex. 3)* | Me | 4-F | 3,5-di-MeO—Ph | Ph | *** |
| 13 (Ex. 4) | Me | - | 3,5-di-MeO—Ph | 2-pyridinyl | *** |
| 14 (Ex. 5) | CH₂Cl | 4-F | 3,5-di-MeO—Ph | Ph | *** |
| 15 | Me | 2,4-di-F | 3,5-di-MeO—Ph | 2-F—Ph | ** |
| 16 | H | 2,4-di-F | 3,5-di-MeO—Ph | 2-F—Ph | ** |
| 18 | Cl | 2,4-di-F | 3,5-di-MeO—Ph | 2-F—Ph | ** |
| 19 | Me | 4-MeO | 3,5-di-MeO—Ph | MeC(═CH₂) | ** |
| 20 | Me | 2,4-di-F | 3,5-di-MeO—Ph | MeC(═CH₂) | ** |
| 21 | Me | 2,6-di-F | 3,5-di-MeO | MeC(═CH₂) | ** |
| 22 | Me | 2,6-di-F | 3,5-di-MeO | 2-F—Ph | ** |
| 23 | Me | 2,4,6-tri-F | 4-Cl—Ph | 2-F—Ph | ** |
| 24 (Ex. 7) | Me | 2,4,6-tri-F | 3,5-di-MeO—Ph | 2-F—Ph | *** |
| 25 | Me | 2,4,6-tri-F | 3-F—Ph | 2-F—Ph | ** |
| 26 (Ex. 11) | MeO | 2,4,6-tri-F | 3,5-di-MeO—Ph | 2-F—Ph | *** |
| 27 (Ex. 12) | H | 2,4,6-tri-F | 3,5-di-MeO—Ph | 2-F—Ph | *** |
| 28 (Ex. 8) | Me | 2,6-di-F, 4-MeO | 3,5-di-MeO—Ph | 2-F—Ph | *** |
| 29 (Ex. 9) | Me | 2,6-di-F, 4-Me₂N(CH₂)₃O | 3,5-di-MeO—Ph | 2-F—Ph | *** |
| 30 | Me | 2,6-di-F, 4-MeNH(CH₂)₃O | 3,5-di-MeO—Ph | 2-F—Ph | ** |
| 31 (Ex. 10) | Me | 2,4,6-tri-F | 2-Cl, 3,5-di-MeO—Ph | 2-F—Ph | *** |
| 32 | Me | 2,4,6-tri-F | 2-Br, 3,5-di-MeO—Ph | 2-F—Ph | ** |
| 33 | Me | 2,4,6-tri-F | 4-Cl, 3,5-di-MeO—Ph | 2-F—Ph | 202.5-209 |
| 34 | Cl | 2,4,6-tri-F | 3,5-di-MeO—Ph | Ph | ** |
| 35 | Me | 2,4,6-tri-F | 3,5-di-MeO—Ph | Ph | ** |
| 36 | Me | 2,4,6-tri-F | 2-Cl, 3,5-di-MeO—Ph | Ph | ** |
| 37 | Me | 2,6-di-F, 4-MeO | 3,5-di-MeO—Ph | Ph | ** |
| 38 (Ex. 13) | Cl | 2,4,6-tri-F, 3-Me₃Si | 3,5-di-MeO—Ph | 2-F—Ph | *** |
| 39 | Cl | 2,4,6-tri-F | 5-F-3-pyridinyl | 2-F—Ph | ** |
| 40 | Cl | 2,4,6-tri-F | 4-MeO-3-pyridinyl | 2-F—Ph | ** |
| 41 | Me | 2,4,6-tri-F | 5-F-3-pyridinyl | 2-F—Ph | ** |
| 42 | Me | 2,4,6-tri-F | 4-MeO-3-pyridinyl | 2-F—Ph | ** |
| 43 | Cl | 2,4,6-tri-F | 2-Cl, 3,5-di-MeO—Ph | 2-F—Ph | ** |
| 44 (Ex. 14) | Cl | 2,4,6-tri-F | 5-MeO-3-pyridinyl | 2-F—Ph | *** |
| 45 (Ex. 6) | Cl | 2,4,6-tri-F | 3,5-di-MeO—Ph | 2-F—Ph | *** |
| 46 | Me | 2,4,6-tri-F | 5-MeO-3-pyridinyl | 2-F—Ph | ** |

A dash (“-”) in the (R⁴)ₘ column indicates m is 0 and hydrogen is present at all positions.
* N-oxide.
** See Index TABLE B for ¹H NMR data.
*** See synthesis example for ¹H NMR data.

INDEX TABLE B

| Com- | |
|---|---|
| pound | ¹H NMR Data (CDCl₃ solution unless indicated otherwise)^{a} |
| 2 | δ 7.4 (t, 1H), 7.3 (m, 1H), 7.16 (t, 1H), 7.1-7.0 (m, 4H), |
| | 6.9 (t, 1H), 6.18 (s, H), 5.94 (s, 2H), 3.48 (s, 6H), 2.61 (s, 3H). |
| 3 | δ 7.4 (m, 2H), 7.28-7.2 (m, 6H), 7.0 (d, 2H), 6.18 (s, 1H), |
| | 5.9 (s, 2H), 3.45 (s, 6H), 2.58 (s, 3H). |
| 4 | δ 7.4 (t, 1H), 7.3-7.2 (m, 4H), 7.1 (t, 1H), 7.0 (m, 2H), |
| | 6.9 (t, 1H), 6.12 (s, 1H), 5.95 (s, 2H), 3.45 (s, 6H), 2.61 (s, 3H). |
| 5 | δ 7.8 (t, 1H), 7.6 (t, 1H), 7.4 (m, 1H), 7.29-7.28 (m, 3H), |
| | 7.0 (d, 2H), 6.14 (s, 1H), 5.9 (s, 2H), 3.48 (s, 6H), 2.60 (s, 3H). |
| 6 | δ 7.4 (m, 2H), 7.3-7.2 (m, 5H), 7.0 (d, 2H), 6.19 (s, 1H), |
| | 5.9 (s, 2H), 3.5 (s, 6H), 2.58 (s, 3H). |
| 7 | δ 7.38 (m, 2H), 7.3-7.2 (m, 3H), 6.95 (d, 2H), 6.83 (d, 2H), |
| | 6.18 (s, 1H), 5.9 (s, 2H), 3.79 (s, 3H), 3.48 (s, 6H), 2.59 (s, 3H). |
| 9 | δ 7.4 (m, 1H), 6.96 (d, 2H), 6.9 (t, 1H), 6.8 (d, 2H), 6.6 (t, 1H), |
| | 6.18 (s, 1H), 5.9 (s, 2H), 3.79 (s, 3H), 3.50 (s, 6H), 2.61 (s, 3H). |
| 10 | δ 7.3 (m, 3H), 7.2 (m, 1H), 7.1-7.0 (m, 4H), 6.1 (s, 1H), |
| | 5.9 (s, 2H), 3.47 (s, 6H), 2.62 (s, 3H). |
| 11 | δ 7.4 (m, 2H), 7.3-7.2 (m, 3H), 7.0 (m, 2H), 6.95 (t, 2H), |
| | 6.18 (s, H), 5.9 (s, 2H), 3.49 (s, 6H), 2.58 (s, 3H). |
| 15 | δ 7.4 (t, 1H), 7.3 (m, 1H), 7.1 (t, 1H), 6.9 (m, 2H), |
| | 6.8 (m, 2H), 6.16 (s, H), 5.99 (s, 2H), 3.51 (s, 6H), 2.60 (s, 3H). |
| 16 | δ 9.2 (s, 1H), 7.4 (t, 1H), 7.3 (m, 1H), 7.18 (t, 1H), 7.08 (m, 1H), |
| | 6.9 (m, 1H), 6.8 (m, 1H), 6.2 (s, 1H), 6.0 (s, 2H), 3.5 (s, 6H). |
| 18 | δ 7.4 (t, 1H), 7.3 (m, 1H), 7.18 (t, 1H), 7.0 (m, 1H), 6.9 (t, 1H), |
| | 6.84 (t, 2H), 6.2 (s, 1H), 6.0 (s, 2H), 3.51 (s, 6H). |
| 19 | δ 6.9 (d, 2H), 6.8 (d, 2H), 6.2 (s, 1H), 6.12 (s, 2H), 5.2 (s, 1H), |
| | 5.1 (s, 1H), 3.78 (s, 3H), 3.63 (s, 6H), 2.53 (s, 3H), 1.91 (s, 3H). |
| 20 | δ 6.9-6.7 (m, 3H), 6.2 (s, 1H), 6.1 (s, 2H), 5.2 (s, 1H), 5.1 (s, 1H), |
| | 3.6 (s, 6H), 2.51 (s, 3H), 1.94 (s, 3H). |
| 21 | δ 7.2 (m, 1H), 6.8 (m, 2H), 6.28 (s, 1H), 6.20 (s, 2H), 5.2 (s, 1H), |
| | 5.1 (s, 1H), 3.65 (s, 6H), 2.53 (s, 3H), 1.96 (s, 3H). |
| 22 | δ 7.4 (t, 1H), 7.3-7.2 (m, 2H), 7.1 (t, 1H), 6.96 (t, 1H), |
| | 6.88 (m, 2H), 6.17 (s, 1H), 6.0 (s, 2H), 3.5 (s, 6H), 2.62 (s, 3H). |
| 23 | δ 7.4 (m, 1H), 7.3 (m, 1H), 7.2 (t, 1H), 7.0 (d, 2H), 6.9 (m, 1H), |
| | 6.84 (d, 2H), 6.6 (t, 2H), 2.63 (s, 3H). |
| 25 | δ 7.4 (m, 1H), 7.3 (m, 1H), 7.18 (m, 1H), 7.0 (m, 1H), |
| | 6.9 (m, 1H), 6.8 (m, 1H), 6.65-6.0 (m, 4H), 2.63 (s, 3H). |
| 30 | δ 7.4 (t, 1H), 7.3 (m, 1H), 7.1 (m, 1H), 6.9 (m, 1H), |
| | 6.4 (d, 2H), 6.19 (s, H), 6.07 (s, 2H), 3.9 (t, 2H), 3.53 (s, 6H), |
| | 2.74 (t, 2H), 2.61 (s, 3H), 2.45 (s, 3H), 1.9 (t, 2H). |
| 32 | δ 7.4 (t, 1H), 7.3 (m, 1H), 7.1 (t, 1H), 6.9 (t, 1H), 6.7-6.6 (m, 2H), |
| | 6.3 (s, H), 6.24 (s, 2H), 3.70 (s, 3H), 3.65 (s, 3H), 2.64 (s, 3H). |
| 34 | δ 7.39 (d, 2H), 7.3-7.2 (m, 3H), 6.6 (t, 2H), 6.2 (s, 1H), |
| | 6.0 (s, 2H), 3.55 (s, 6H). |
| 35 | δ 7.4 (d, 2H), 7.3-7.2 (m, 3H), 6.6 (t, 2H), 6.2 (s, 1H), |
| | 6.0 (d, 2H), 3.55 (s, 6H), 2.59 (s, 3H). |
| 36 | δ 7.4 (d, 2H), 7.3-7.2 (m, 3H), 6.6 (m, 2H), 6.3 (s, 1H), |
| | 6.1 (s, 1H), 3.74 (s, 3H), 3.61 (s, 3H), 2.6 (s, 3H). |
| 37 | δ 7.4 (d, 2H), 7.3-7.2 (m, 3H), 6.4 (d, 2H), 6.2 (s, 1H), |
| | 6.0 (d, 2H), 3.77 (s, 3H), 3.54 (s, 6H), 2.58 (s, 3H). |
| 39 | δ 8.3 (d, 1H), 7.99 (s, 1H), 7.6 (m, 1H), 7.4 (m, 1H), |
| | 7.2 (m, 1H), 7.0 (d, 1H), 6.9 (t, 1H), 6.7 (m, 2H). |
| 40 | δ 7.7 (d, 1H), 7.5 (t, 1H), 7.3 (m, 1H), 7.2 (m, 1H), |
| | 7.1 (d, 1H), 6.9 (t, 1H), 6.6 (m, 2H), 6.4 (d, 1H), 3.82 (s, 3H). |
| 41 | δ 8.2 (d, 1H), 7.9 (s, 1H), 7.59 (m, 1H), 7.3 (m, 1H), |
| | 7.2 (m, 1H), 7.0 (d, 1H), 6.9 (t, 1H), 6.69 (m, 2H), 2.66 (s, 3H). |
| 42 | δ 7.7 (d, 1H), 7.5 (t, 1H), 7.3 (m, 1H), 7.2 (m, 1H), 7.1 (d, 1H), |
| | 6.9 (t, 1H), 6.68 (m, 2H), 6.4 (d, 1H), 3.82 (s, 3H), 2.63 (s, 3H). |
| 43 | δ 7.4 (t, 1H), 7.3 (m, 1H), 7.1 (t, 1H), 6.9 (m, 1H), 6.7-6.6 (m, 2H), |
| | 6.30 (s, 1H), 6.25 (s, 1H), 3.71 (s, 3H), 3.63 (s, 3H). |
| 46 | δ 8.0 (d, 1H), 7.7 (s, 1H), 7.5 (m, 1H), 7.3 (m, 1H), 7.2 (m, 1H), |
| | 6.9 (t, 1H), 6.7 (s, 1H), 6.6 (m, 2H), 3.60 (s, 3H), 2.65 (s, 3H). |

^{a1}H NMR data are in ppm downfield from tetramethylsilane.
Couplings are designated by (s)-singlet, (d)-doublet, (t)-triplet, (m)-multiplet.

### BIOLOGICAL EXAMPLES OF THE INVENTION

General protocol for preparing test suspensions for Tests A-F: The test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in Tests A-F. The test suspensions were sprayed to the point of run-off on the test plants. All results are for 200 ppm (equivalent to a rate of 500 g/ha) except where followed by “*” which indicates 40 ppm.

### Test A

The test suspension was sprayed to the point of run-off on wheat seedlings. The following day the seedlings were inoculated with a spore dust of Erysiphe graminis, (the causal agent of wheat powdery mildew) and incubated in a growth chamber at 20° C. for 8 days, after which time visual disease ratings were made.

### Test B

The test suspension was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time visual disease ratings were made.

### Test C

The test suspension was sprayed to the point of run-off on wheat seedlings. The following day the seedlings were inoculated with a spore suspension of Septoria tritici (the causal agent of wheat leaf blotch) and incubated in saturated atmosphere at 20° C. for 48 h, and moved to a growth chamber at 20° C. for 19 additional days, after which time visual disease ratings were made.

### Test D

The test suspension was sprayed to the point of run-off on wheat seedlings. The following day the seedlings were inoculated with a spore suspension of Septoria nodorum (the causal agent of wheat glume blotch) and incubated in a saturated atmosphere at 20° C. for 48 h, and then moved to a growth chamber at 20° C. for 7 days, after which time visual disease ratings were made.

### Test E

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time visual disease ratings were made.

### Test F

The test suspension was sprayed to the point of run-off on tomato seedlings. The following day the seedlings were inoculated with a spore suspension of Botrytis cinerea (the causal agent of tomato Botrytis) and incubated in saturated atmosphere at 20° C. for 48 h, and then moved to a growth chamber at 24° C. for 3 days, after which time visual disease ratings were made.

Results for Tests A-F are given in Table A. In the table, a rating of 100 indicates 100% disease control and a rating of 0 indicates no disease control (relative to the controls). A dash (-) indicates no test results. All results are for 200 ppm except where followed by “*”, which indicates 40 ppm.

TABLE A

| Cmpd No. | Test A | Test B | Test C | Test D | Test E | Test F |
|---|---|---|---|---|---|---|
| 1 | 85 | 99 | 100 | 0 | 72 | 98 |
| 2 | 97 | 99 | 100 | 0 | 99 | 99 |
| 3 | 52 | 86 | 87 | 0 | 0 | 52 |
| 4 | 82 | 96 | 84 | 0 | 28 | 94 |
| 5 | 91 | 86 | 94 | 0 | 0 | 82 |
| 6 | 79 | 97 | 97 | 0 | 36 | 67 |
| 7 | 95 | 100 | 100 | 90 | 96 | 97 |
| 8 | 97 | 100 | 100 | 95 | 99 | 98 |
| 9 | 84 | 91 | 100 | 60 | 81 | 98 |
| 10 | 97 | 99 | 99 | 0 | 88 | 95 |
| 11 | 93 | 68 | 70 | 0 | 0 | 0 |
| 12 | 62 | 100 | 99 | 0 | 94 | 100 |
| 13 | 0 | 41 | 0 | 0 | 0 | 0 |
| 14 | 11 | 91 | 77 | 0 | 0 | 99 |
| 15 | 100 | 100 | 99 | 98 | 100 | 98 |
| 16 | 0 | 96 | 70 | 0 | 0 | 99 |
| 18 | 95 | 100 | 98 | 100 | 100 | 95 |
| 19 | 61 | 98 | 98 | 0 | 100 | 98 |
| 20 | 96 | 100 | 96 | 0 | 100 | 99 |
| 21 | 94* | 99* | 98* | 0* | 98* | 98* |
| 22 | 99* | 100* | 97* | 84* | 100* | 95* |
| 23 | 99 | 99 | 97 | 0 | 96 | 86 |
| 24 | 100 | 100 | 96 | 100 | 100 | 94 |
| 25 | 99 | 100 | 96 | 100 | 100 | 98 |
| 26 | 90 | 100 | 99 | 100 | 95 | 99 |
| 27 | 89 | 100 | 99 | 84 | 99 | 100 |
| 28 | 85* | 100* | 97* | 100* | 100* | 100* |
| 29 | 92 | 100 | 98 | 92 | 99 | 99 |
| 30 | 89 | 100 | 99 | 98 | 98 | 100 |
| 31 | 97* | 100* | 99* | 100* | 100* | 100* |
| 32 | 90* | 100* | 98* | 99* | 99* | 100* |
| 33 | 0* | 97* | 95* | 0* | 61* | 99* |
| 34 | 99 | 100 | 100 | 93 | 99 | 100 |
| 35 | 90* | 100* | 100* | 95 | 99 | 99 |
| 36 | 98* | 99* | 99* | 100* | 99* | 99* |
| 37 | 69* | 100* | 98* | 99* | 99* | 99* |
| 38 | 0* | 98* | 94* | 0* | 26* | 99* |
| 39 | 100* | 100* | 100* | 100* | 100* | 100* |
| 40 | 73* | 100* | 100* | 60* | 93* | 100* |
| 41 | 97* | 100* | 100* | 98* | 98* | 100* |
| 42 | 78* | 99* | 100* | 0* | 41* | 100* |
| 43 | 99* | 100* | 97* | 100* | 99* | 100* |
| 44 | 100* | 99* | 99* | 100* | 100* | 100* |
| 45 | 100* | 100* | 99* | 100* | 100* | 100* |
| 46 | 97* | 100* | 99* | 0* | 100* | 100* |

What is claimed is:

1. A compound selected from Formula 1, N-oxides, and salts thereof, 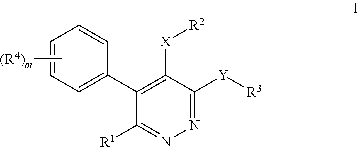
wherein
R¹ is H, halogen, cyano, hydroxy, amino, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ alkoxycarbonyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkylamino or C₂-C₄ dialkylamino;
each X and Y is independently CH₂ or a direct bond;
R² is a phenyl ring optionally substituted with up to 5 substituents independently selected from R⁵; or a 3- to 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁵ on carbon atom ring members and R^{5a} on nitrogen atom ring members;
R³ is a phenyl ring optionally substituted with up to 5 substituents independently selected from R⁶; or a 3- to 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 5 substituents independently selected from R⁶ on carbon atom ring members and R^{6a} on nitrogen atom ring members;
each R⁴, R⁵ and R⁶ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₁-C₆ alkylthio, C₁-C₆ haloalkylthio, C₂-C₆ alkylcarbonylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₃-C₉ trialkylsilyl or —Z—V—W;
each Z is independently O, S(═O)ₙ, NR⁸ or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each W is independently NR^{9a}R^{9b}, OR¹⁰ or S(═O)ₙR¹⁰;
each R^{5a} and R^{6a} is independently cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₁-C₆ alkylthio, C₁-C₆ haloalkylthio, C₁-C₆ alkylsulfonyl, C₁-C₆ haloalkylsulfonyl or C₃-C₉ trialkylsilyl; or
one pair of R⁴ substituents, one pair of R⁵ or R^{5a} substituents, or one pair of R⁶ or R^{6a} substituents attached to adjacent ring atoms are each independently taken together with the atoms to which they are attached to form a 5- to 7-membered fused ring, each fused ring containing ring members selected from carbon atoms and up to 4 heteroatoms independently selected from up to 2 oxygen, up to 2 sulfur and up to 3 nitrogen atoms, and optionally substituted with up to 3 substituents independently selected from the group consisting of halogen, cyano, nitro, C₁-C₂ alkyl and C₁-C₂ alkoxy on carbon atom ring members and from the group consisting of cyano, C₁-C₂ alkyl and C₁-C₂ alkoxy on nitrogen atom ring members; or
one pair of R⁵ substituents, or one pair of R⁶ substituents attached to the same ring atom are each independently taken together with the atom to which they are attached to form a 5- to 7-membered spirocyclic ring, each spirocyclic ring containing ring members selected from carbon atoms and up to 4 heteroatoms independently selected from up to 2 oxygen, up to 2 sulfur and up to 3 nitrogen atoms, and optionally substituted with up to 3 substituents independently selected from the group consisting of halogen, cyano, nitro, C₁-C₂ alkyl and C₁-C₂ alkoxy on carbon atom ring members and from the group consisting of cyano, C₁-C₂ alkyl and C₁-C₂ alkoxy on nitrogen atom ring members;
each R⁷ is independently H or C₁-C₆ alkyl;
each R⁸ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{9a} and R^{9b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{9a} and R^{9b} attached to the same nitrogen atom are taken together with the nitrogen atom to form a 3- to 6-membered heterocyclic ring, the ring optionally substituted with up to 5 substituents independently selected from R¹¹;
each R¹⁰ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹¹ is independently halogen, C₁-C₆ alkyl, C₁-C₆ haloalkyl or C₁-C₆ alkoxy;
m is 0, 1, 2, 3, 4 or 5;
each n is independently 0, 1 or 2; and
p and q are independently 0, 1 or 2 in each instance of S(═O)ₚ(═NR⁷)_{q}, provided that the sum of p and q is 0, 1 or 2;
provided that when R² and R³ are phenyl rings, then at least one of R² and R³ is substituted with a substituent other than hydrogen.

2. A compound of claim 1 wherein:
R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio;
R² is a phenyl ring optionally substituted with up to 3 substituents independently selected from R⁵; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁵ on carbon atom ring members and R^{5a} on nitrogen atom ring members;
R³ is a phenyl ring optionally substituted with up to 3 substituents independently selected from R⁶; or a 5- or 6-membered heterocyclic ring containing ring members selected from carbon atoms and up to 4 heteroatoms independently selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁷)_{q}, the heterocyclic ring optionally substituted with up to 3 substituents independently selected from R⁶ on carbon atom ring members and R^{6a} on nitrogen atom ring members;
each R⁴, R⁵ and R⁶ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio, C₁-C₆ haloalkylthio or —Z—V—W;
each R^{5a} and R^{6a} is independently C₁-C₃ alkyl or C₁-C₃ haloalkyl; and
m is 0, 1, 2 or 3.

3. The compound of claim 2 wherein:
R¹ is halogen, cyano, C₁-C₂ alkyl or C₁-C₂ alkoxy;
R² is a phenyl or pyridinyl ring optionally substituted with up to 3 substituents independently selected from R⁵;
R³ is a phenyl or pyridinyl ring optionally substituted with up to 3 substituents independently selected from R⁶;
each R⁴, R⁵ and R⁶ is independently halogen, C₁-C₆ alkyl, C₂-C₆ alkenyl, C₁-C₆ haloalkyl, C₁-C₆ alkoxy or —Z—V—W;
each X and Y is a direct bond;
each Z is independently O or NH;
each V is C₂-C₄ alkylene;
each W is independently NR^{9a}R^{9b} or OR¹⁰;
each R^{9a} and R^{9b} is independently H, C₁-C₂ alkyl or C₁-C₂ haloalkyl; and
each R¹⁰ is methyl.

4. The compound of claim 3 wherein:
R¹ is chloro, methyl or methoxy;
R² is a phenyl ring optionally substituted with up to 3 substituents independently selected from R⁵;
R³ is a phenyl ring optionally substituted with up to 3 substituents independently selected from R⁶;
each R⁴, R⁵ and R⁶ is independently halogen, C₁-C₃ alkyl, C₂-C₃ alkenyl, C₁-C₃ haloalkyl or C₁-C₃ alkoxy; and
the R² ring is substituted with at least one substituent at a meta position and the R³ ring is substituted with at least one substituent at an ortho or para position.

5. The compound of claim 4 wherein:
each R⁴, R⁵ and R⁶ is independently halogen, C₁-C₃ alkyl or C₁-C₃ alkoxy.

6. The compound of claim 5 wherein:
each R⁴ and R⁵ is independently Cl, F or methoxy; and
the R² ring is substituted with at least two substituents at the meta positions and the R³ ring is substituted with at least one substituent at an ortho or para position.

7. A compound of claim 1 which is selected from the group consisting of:
4-(3,5-dimethoxyphenyl)-3-(2-fluorophenyl)-6-methyl-5-(2,4,6-trifluorophenyl)pyridazine;
4-(2,6-difluoro-4-methoxyphenyl)-5-(3,5-dimethoxyphenyl)-6-(2-fluorophenyl)-3-methylpyridazine;
4-(2-chloro-3,5-dimethoxyphenyl)-3-(2-fluorophenyl)-6-methyl-5-(2,4,6-trifluorophenyl)pyridazine;
4-(3,5-dimethoxyphenyl)-3-(2-fluorophenyl)-6-methoxy-5-(2,4,6-trifluorophenyl)pyridazine;
4-(3,5-dimethoxyphenyl)-3-(2-fluorophenyl)-5-(4-fluorophenyl)-6-methylpyridazine;
4-(3,5-dimethoxyphenyl)-5-(4-methoxyphenyl)-6-methyl-3-phenylpyridazine;
3-(2,4-difluorophenyl)-4-(3,5-dimethoxyphenyl)-5-(4-methoxyphenyl)-6-methylpyridazine;
4-(2,4-difluorophenyl)-5-(3,5-dimethoxyphenyl)-6-(2-fluorophenyl)-3-methylpyridazine;
3-chloro-4-(2,4-difluorophenyl)-5-(3,5-dimethoxyphenyl)-6-(2-fluorophenyl)pyridazine;
3-(2-fluorophenyl)-4-(3-fluorophenyl)-6-methyl-5-(2,4,6-trifluorophenyl)pyridazine;
3-[4-[5-(3,5-dimethoxyphenyl)-6-(2-fluorophenyl)-3-methyl-4-pyridazinyl]-3,5-difluorophenoxy]-N-methyl-1-propanamine;
4-(3,5-dimethoxyphenyl)-6-methyl-3-phenyl-5-(2,4,6-trifluorophenyl)pyridazine;
4-(2-chloro-3,5-dimethoxyphenyl)-6-methyl-3-phenyl-5-(2,4,6-trifluorophenyl)pyridazine;
5-(2,6-difluoro-4-methoxyphenyl)-4-(3,5-dimethoxyphenyl)-6-methyl-3-phenylpyridazine; and
4-(3,5-dimethoxyphenyl)-3-(2-fluorophenyl)-5-(4-methoxyphenyl)-6-methylpyridazine.

8. A compound of claim 1 which is selected from the group consisting of:
3-chloro-4-(2,4-difluorophenyl)-5-(3,5-dimethoxyphenyl)-6-(2-fluorophenyl)pyridazine.

9. A fungicidal composition comprising (a) a compound of claim 1; and (b) at least one other fungicide.

10. A fungicidal composition comprising (a) a fungicidally effective amount of a compound of claim 1; and (b) at least one additional component selected from the group consisting of surfactants, solid diluents and liquid diluents.

11. A method for controlling plant diseases caused by fungal plant pathogens comprising applying to the plant or portion thereof, or to the plant seed, a fungicidally effective amount of a compound of claim 1.
